(19) United States
(12) Patent Application Publication
(10) Pub. No.: US 2025/0313580 A1
(43) Pub. Date: Oct. 9, 2025

(54) Synthesis of fluorosilyl compounds

(71) Applicant: Blue Earth Therapeutics Limited, Oxford (GB)

(72) Inventors: Thonan Lee Ford, Wolverhampton (GB); Alexander James Floy Thomas, Wolverhampton (GB); Sabitur Rahman, Oxford (GB)

(73) Assignee: Blue Earth Therapeutics Limited, Oxford (GB)

(21) Appl. No.: 18/866,804

(22) PCT Filed: May 19, 2023
(86) PCT No.: PCT/GB2023/051326

§ 371 Date: Nov. 18, 2024

(30) Foreign Application Priority Data
May 19, 2022 (GB) 2207372.0

(51) Int. Cl. C07F 7/12; A61K 51/04; C07F 5/00

(52) U.S. Cl. CPC C07F 7/122; A61K 51/0402; C07F 5/003

(57) ABSTRACT

The disclosures herein relate to methods of synthesising a compound of formula (1):

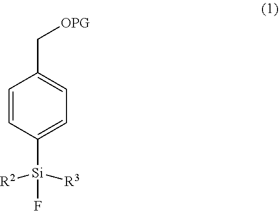

wherein PG, R² and R³ are as defined herein, and the use of said methods in the manufacture of conjugates that bind to PSMA.

### FIELD OF THE INVENTION

The invention relates to methods for the synthesis of fluorosilyl compounds and more particularly compounds comprising silicon fluoride acceptor (SiFA) moieties and derivatives thereof.

### BACKGROUND OF THE INVENTION

4-(di-alkylfluorosilyl)benzoic acids, which are an example of silicon fluoride acceptors (SiFAs), are fluorine-containing molecules used in the synthesis of fluorine-labelled molecules, especially where it is desirable for the fluorine to be enriched with the 18F radioisotope. The 18F radioisotope is widely used in positron emission tomography (PET).

SiFAs have been attached to prostate-specific membrane antigen (PSMA) targeting molecules to form PSMA-SiFA conjugates. The use of these conjugates in combination with 18F PET imaging enables the visualization of prostate cancers. The addition of radiotherapeutic moieties to these PSMA-SiFA conjugates, specifically by the addition of chelating groups capable of chelating radioisotopes, enables visualization of the relevant area together with targeted radiotherapy. This dual functionality reduces the chance of off-target radiation damage. WO2019/020831, WO2020/157177 and WO2020/157184 disclose PSMA-SiFA conjugates.

Known procedures for the synthesis of SiFA 4-(di-tert-butylfluorosilyl)benzoic acid, are disclosed in Chem. Eur. J. 2009, 15, 2140-2147 and WO2020/157128, for example.

However, there remains a need for improved methods of synthesis of SiFAs, including 4-(di-alkylfluorosilyl)benzoic acids and precursors thereof. In particular, synthetic procedures allowing for the preparation of such compounds in high yield and with high purity are desired.

### SUMMARY OF THE INVENTION

The invention provides a method of preparing a compound of formula (1) comprising reacting a compound of formula (2) with an alkyllithium reagent of formula (3) and a compound of formula (4):

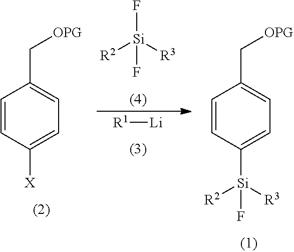

wherein PG is a protecting group;
X is Br, Cl or I;
R² and R³ are independently linear or branched C₁₋₁₀ alkyl;
R¹ is linear C₁₋₆ alkyl; and
wherein the alkyllithium reagent of formula (3) is added to a mixture comprising the compounds of formula (2) and (4).

In the methods of the invention the linear alkyllithium reagent of formula (3) is added to a mixture comprising the compounds of formula (2) and (4). This one-pot procedure is in contrast to known procedures which typically require step-wise addition of tert-butyllithium to the compound of formula (2) at cryogenic temperatures, followed by later addition of the compound of formula (4).

The methods of the invention have been found to provide significantly improved yields of compounds of formula (1) as compared to analogous known procedures. Furthermore, the methods of the invention do not require the use of tert-butyllithium, nor do they require cryogenic reaction conditions. Linear alkyllithium reagents, such as n-butyllithium, may be used under milder reaction temperatures. As such the methods of the invention are inherently safer and better suited to scale-up.

### DESCRIPTION OF THE INVENTION

The invention provides a method of preparing a compound of formula (1) comprising reacting a compound of formula (2) with an alkyllithium reagent of formula (3) and a compound of formula (4):

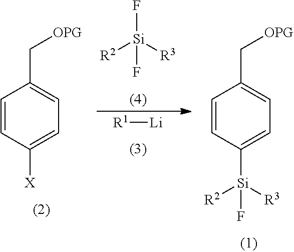

wherein PG is a protecting group;
X is Br, Cl or I;
R² and R³ are independently linear or branched C₁₋₁₀ alkyl;
R¹ is linear C₁₋₆ alkyl; and
wherein the alkyllithium reagent of formula (3) is added to a mixture comprising the compounds of formula (2) and (4).

In the methods and compounds herein, PG is a protecting group. PG can be an acid-labile protecting group. PG can be a silyl ether protecting group. PG can be selected from trimethylsilyl (TMS), triethylsilyl(TES), tert-butyldimethylsilyl(TBDMS), tert-butyldiphenylsilyl(TBDPS), triisopropylsilyl(TIPS) and di-tert-butylmethylsilyl(DTBMS). PG can be tert-butyldimethylsilyl (TBDMS).

In the methods and compounds herein, X is Br, Cl or I. X can be Br. X can be Cl. X can be I.

The compound of formula (2) can be a compound of formula (2a), (2b) or (2c):

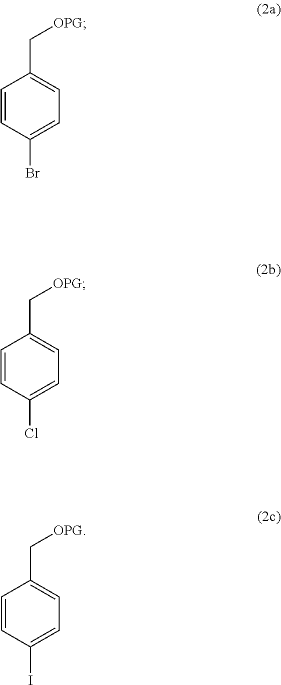

The compound of formula (2) can be a compound of formula (2a):

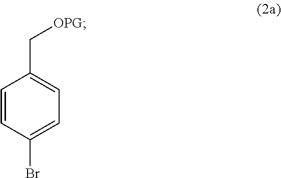

The compound of formula (2) can be a compound of formula (2d), (2e) or (2f):

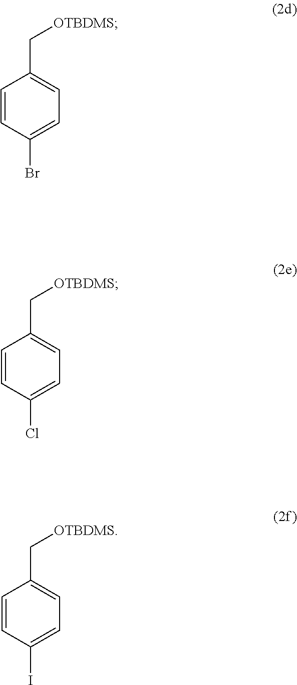 [SMILES: CC(C)(C)[Si](C)(C)OCc1ccc(Br)cc1 ; CC(C)(C)[Si](C)(C)OCc1ccc(Cl)cc1 ; CC(C)(C)[Si](C)(C)OCc1ccc(I)cc1]

The compound of formula (2) can be a compound of formula (2d):

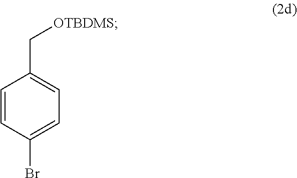 [SMILES: CC(C)(C)[Si](C)(C)OCc1ccc(Br)cc1]

In the methods and compounds herein, R¹ is linear C₁₋₆ alkyl. Hence the alkyllithium reagent of formula (3) is a linear C₁₋₆ alkyllithium reagent. R¹ can be linear C₁₋₅ alkyl. R¹ can be linear C₁₋₄ alkyl. R¹ can be selected from methyl, ethyl, n-propyl, n-butyl, n-pentyl and n-hexyl. The alkyllithium reagent of formula (3) can be selected from methyllithium, ethyllithium, n-propyllithium, n-butyllithium, n-pentyllithium and n-hexylithium. R¹ can be n-butyl. The alkyllithium reagent of formula (3) can be n-butyllithium.

In the methods and compounds herein, R² and R³ are independently linear or branched C₁₋₁₀ alkyl. R² and R³ can independently be linear or branched C₁₋₆ alkyl. R² and R³ can independently be linear or branched C₁₋₄ alkyl. R² and R³ can independently be linear or branched C₃₋₁₀ alkyl. R² and R³ can independently be linear or branched C₃₋₆ alkyl. R² and R³ can independently be linear or branched C₃₋₄ alkyl. R² and R³ can independently be linear C₁₋₁₀ alkyl. R² and R³ can independently be linear C₁₋₆ alkyl. R² and R³ can independently be linear C₁₋₄ alkyl. R² and R³ can independently be branched C₃₋₁₀ alkyl. R² and R³ can independently be branched C₃₋₆ alkyl. R² and R³ can independently be branched C₃₋₄ alkyl. R² and R³ can independently be selected from methyl, ethyl, isopropyl, n-propyl, n-butyl, sec-butyl and tert-butyl. R² and R³ can both be tert-butyl.

In the methods and compounds herein, R² can be linear or branched C₁₋₁₀ alkyl. R² can be linear or branched C₁₋₆ alkyl. R² can be linear or branched C₁₋₄ alkyl. R² can be linear or branched C₃₋₁₀ alkyl. R² can be linear or branched C₃₋₆ alkyl. R² can be linear or branched C₃₋₄ alkyl. R² can be linear C₁₋₁₀ alkyl. R² can be linear C₁₋₆ alkyl. R² can be linear C₁₋₄ alkyl. R² can be branched C₃₋₁₀ alkyl. R² can be branched C₃₋₆ alkyl. R² can be branched C₃₋₄ alkyl. R² can be selected from methyl, ethyl, isopropyl, n-propyl, n-butyl, sec-butyl and tert-butyl. R² can be tert-butyl.

In the methods and compounds herein, R³ can be linear or branched C₁₋₁₀ alkyl. R³ can be linear or branched C₁₋₆ alkyl. R³ can be linear or branched C₁₋₄ alkyl. R³ can be linear or branched C₃₋₁₀ alkyl. R³ can be linear or branched C₃₋₆ alkyl. R³ can be linear or branched C₃₋₄ alkyl. R³ can be linear C₁₋₁₀ alkyl. R³ can be linear C₁₋₆ alkyl. R³ can be linear C₁₋₄ alkyl. R³ can be branched C₃₋₁₀ alkyl. R³ can be branched C₃₋₆ alkyl. R³ can be branched C₃₋₄ alkyl. R³ can be selected from methyl, ethyl, isopropyl, n-propyl, n-butyl, sec-butyl and tert-butyl. R³ can be tert-butyl.

The compound of formula (4) can be a compound of formula (4a):

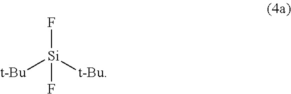 [SMILES: CC(C)(C)[Si](F)(F)C(C)(C)C]

The compound of formula (1) can be a compound of formula (1a):

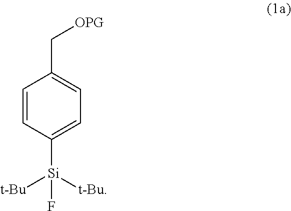

The compound of formula (1) can be a compound of formula (1b):

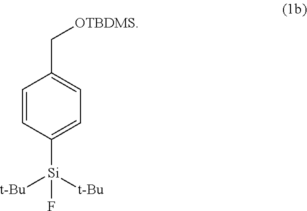 [SMILES: CC(C)(C)[Si](C)(C)OCc1ccc([Si](F)(C(C)(C)C)C(C)(C)C)cc1]

The method of the invention may comprise reacting a compound of formula (2d) with n-butyllithium (n-BuLi) and a compound of formula (4a) to form a compound of formula (1b):

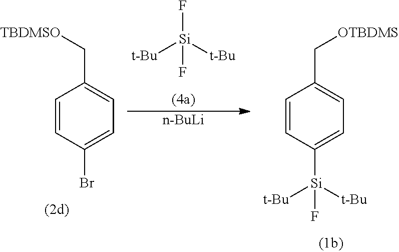

wherein the n-BuLi is added to a mixture comprising the compounds of formula (2d) and (4a).

In the methods herein, the mixture comprising the compounds of formula (2) and (4) or (2a) and (4a) or (2d) and (4a) may be maintained at a temperature of between −10° C. and 30° C. during addition of the alkyllithium reagent. The mixture comprising the compounds of formula (2) and (4) or (2a) and (4a) or (2d) and (4a) may be maintained at a temperature of between −10° C. and 20° C. during addition of the alkyllithium reagent. The mixture comprising the compounds of formula (2) and (4) or (2a) and (4a) or (2d) and (4a) may be maintained at a temperature of between −5° C. and 25° C. during addition of the alkyllithium reagent. The mixture comprising the compounds of formula (2) and (4) or (2a) and (4a) or (2d) and (4a) may be maintained at a temperature of between −5° C. and 20° C. during addition of the alkyllithium reagent. The mixture comprising the compounds of formula (2) and (4) or (2a) and (4a) or (2d) and (4a) may be maintained at a temperature of between −5° C. and 15° C. during addition of the alkyllithium reagent. The mixture comprising the compounds of formula (2) and (4) or (2a) and (4a) or (2d) and (4a) may be maintained at a temperature of between −5° C. and 10° C. during addition of the alkyllithium reagent. The mixture comprising the compounds of formula (2) and (4) or (2a) and (4a) or (2d) and (4a) may be maintained at a temperature of between −5° C. and 5° C. during addition of the alkyllithium reagent. The mixture comprising the compounds of formula (2) and (4) or (2a) and (4a) or (2d) and (4a) may be maintained at a temperature of between 0° C. and 25° C. during addition of the alkyllithium reagent. The mixture comprising the compounds of formula (2) and (4) or (2a) and (4a) or (2d) and (4a) may be maintained at a temperature of between 0° C. and 20° C. during addition of the alkyllithium reagent. The mixture comprising the compounds of formula (2) and (4) or (2a) and (4a) or (2d) and (4a) may be maintained at a temperature of between 0° C. and 15° C. during addition of the alkyllithium reagent. The mixture comprising the compounds of formula (2) and (4) or (2a) and (4a) or (2d) and (4a) may be maintained at a temperature of between 0° C. and 10° C. during addition of the alkyllithium reagent. The mixture comprising the compounds of formula (2) and (4) or (2a) and (4a) or (2d) and (4a) may be maintained at a temperature of between 0° C. and 5° C. during addition of the alkyllithium reagent.

In the methods herein, 1.0 to 1.5 molar equivalents of alkyllithium reagent may be used relative to the compound of formula (2), (2a), (2b), (2c), (2d), (2e) or (2f). 1.0 to 1.3 molar equivalents of alkyllithium reagent may be used relative to the compound of formula (2), (2a), (2b), (2c), (2d), (2e) or (2f). 1.1 to 1.2 molar equivalents of alkyllithium reagent may be used relative to the compound of formula (2), (2a), (2b), (2c), (2d), (2e) or (2f). 1.15 molar equivalents of alkyllithium reagent may be used relative to the compound of formula (2), (2a), (2b), (2c), (2d), (2e) or (2f).

In the methods herein, 1.0 to 1.5 molar equivalents of the compound of formula (4) or (4a) may be used relative to the compound of formula (2), (2a), (2b), (2c), (2d), (2e) or (2f). 1.0 to 1.3 molar equivalents of the compound of formula (4) or (4a) may be used relative to the compound of formula (2), (2a), (2b), (2c), (2d), (2e) or (2f). 1.1 to 1.2 molar equivalents of the compound of formula (4) or (4a) may be used relative to the compound of formula (2), (2a), (2b), (2c), (2d), (2e) or (2f). 1.15 molar equivalents of the compound of formula (4) or (4a) may be used relative to the compound of formula (2), (2a), (2b), (2c), (2d), (2e) or (2f).

In the methods used to prepare compounds of formula (1), (1a) or (1b) herein, an aprotic solvent may be used. A polar aprotic solvent may be used. The solvent used in may be tetrahydrofuran (THF).

In the methods herein, it will be appreciated that the alkyllithium reagent will be added to the reaction mixture as a solution in an aprotic solvent, for example as a solution in hexanes, for example as a 2.5M solution in hexanes.

The methods herein may comprise a further step of deprotecting the compound of formula (1), (1a) or (1b) using an acid to obtain a compound of formula (5) or (5a):

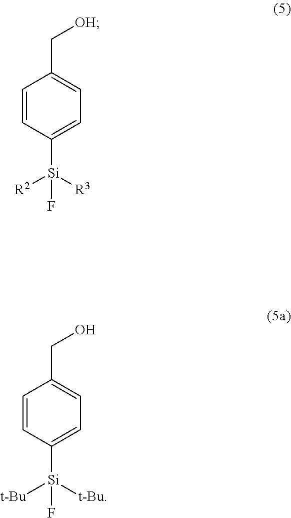

The acid used in the deprotection step may be any acid suitable for deprotection. The acid used may be hydrochloric acid.

The methods herein may comprise a further step of oxidising the compound of formula (5) or (5a) to obtain a compound of formula (6) or (6a):

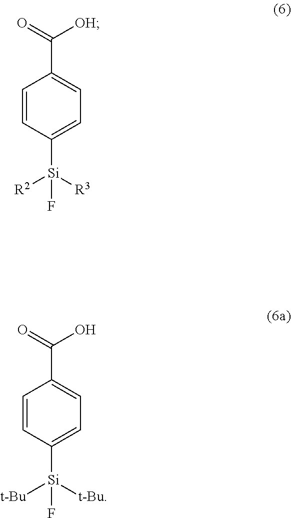

The oxidizing agent used in the oxidation step may be any suitable oxidizing agent. The oxidizing agent may be potassium permanganate (KMnO₄). The oxidation reaction may further comprise us of NaH₂PO₄. The oxidation reaction may further comprise us of NaH₂PO₄.HCl. The oxidation reaction may be followed by a trituration purification step. The trituration purification step may be used to remove undesired oxidation side-products, for example compounds of formula (7), (7a), (7b) or (7c):

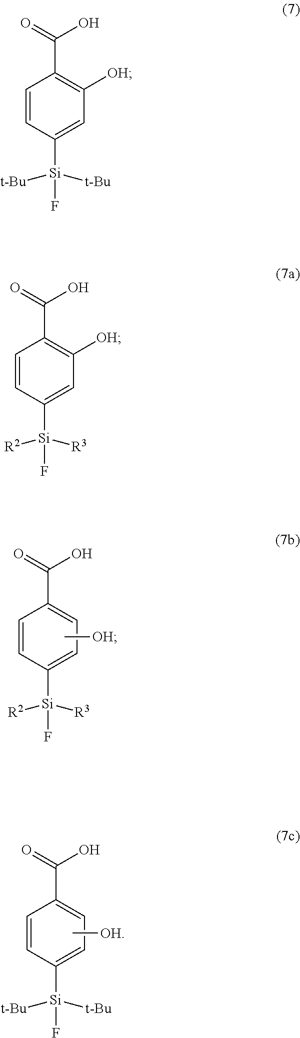

The trituration purification step may make use of a heptane: Et₂O solvent system. The trituration purification step may make use of a 25:1 heptane: Et₂O solvent system.

The trituration purification step may comprise the following steps:
Stirring the crude material comprising the compound of formula (6) or (6a) at elevated temperatures, for example at about 65° C., in 25:1 heptane: Et₂O until dissolved, for example, for up to 30 mins.
Allowing the resultant mixture to cool to r.t. with stirring and then cooling to 0° C. with ice, for example, over about 20 mins.
Filtering the crystallised product and washing the filter cake one or more times with ice-cold 25:1 heptane: Et₂O followed by n-pentane.
Drying the resultant product under vacuum at room temperature to constant weight.

Also provided is a method of synthesising a compound of formula (I) comprising the step of oxidising a compound of formula (II) with potassium permanganate

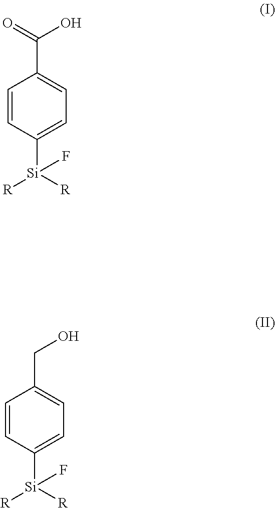

wherein each R is independently a C₁ to C₆ alkyl group.

The compound of formula (I) may be used in the manufacture of a radiopharmaceutical. In some embodiments, the compound of formula (I) may be used in the manufacture of a radiopharmaceutical wherein the fluorine atoms of a compound of formula (I), or a derivative, or subsequent product made thereof, are enriched with the fluorine-18 isotope. Herein disclosed, molecules depicted herein with fluorine atoms should be understood to include embodiments where the fluorine atoms are enriched (i.e. above natural abundance) with the fluorine-18 isotope.

The oxidation step described herein uses a single step oxidation, and avoids the use of pyridinium chlorochromate (a well know toxic carcinogenic compound). In prior art methods that make use of pyridinium chlorochromate, extensive purification must be carried out to ensure that all chromium compounds, and any of their byproducts, are removed before the oxidation product can be safely used in subsequent synthetic steps, and in turn to ensure that final products can be safely administered in a pharmaceutical composition.

The method of synthesising a compound of formula (I) may additionally comprise the step of synthesising the compound of formula (II) by deprotecting a compound of formula (III) using an acid

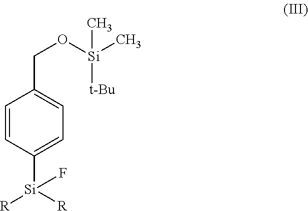

The acid used to deprotect the compound of formula (III) may be hydrochloric acid.

The compound of formula (II), (5) or (5a) may be substantially purified prior to oxidation with potassium permanganate. The compound of formula (II), (5) or (5a) may be substantially purified by column chromatography prior to oxidation with potassium permanganate.

It has been found that the purification of the compound of formula (II), (5) or (5a) prior to oxidation to a compound of formula (I), (6) or (6a) beneficially reduces the presence of undesirable byproducts. Without wishing to be bound by theory, fewer oxidation byproducts are produced where the compound of formula (II), (5) or (5a) is purified prior to the oxidation step, and so purification of the compound of formula (I), (6) or (6a) is ultimately improved.

The method may additionally comprise the step of synthesising the compound of formula (III) by reacting the compound of formula (V), e.g. 4-bromophenyl) methoxy-tert-butyl-dimethyl-silane (a compound with a TBDMS protecting group), with an alkyllithium reagent and a compound of formula (IV)

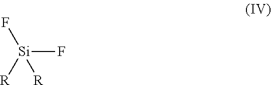

wherein R is defined hereinabove.

It should be understood that other alcohol protecting groups, other than the TBDMS protecting group, are considered within the scope of the invention. In particular when the protecting group is removable under acid conditions (e.g. removable with HCl in alcohol).

The synthesis of the compound of formula (III) may be carried out at a temperature between-10° C. and 30° C., optionally between −5° C. and 25° C., further optionally between 0° C. and 20° C.

The alkyllithium reagent may be n-butyllithium. Advantageously, unlike prior art methods, the use of n-butyllithium avoids the use of tert-butyllithium. Tert-butyllithium is a very reactive compound (pyrophoric, flammable, toxic and corrosive), and so must be handled with extreme care. For safety reasons, the prior art methods prescribe that the tert-butyllithium reaction, using a two fold excess of tert-butyllithium, is carried out in isolation, the reagents are added dropwise, and that the reaction is cooled with dry ice.

Advantageously, herein disclosed, n-butlylithium can be used in the lithium-halogen exchange reaction. Because n-butyllithium is much less reactive and not highly pyrophoric, the lithium-halogen exchange reaction can be cooled with ice water. It has also been found that a roughly equimolar ratio of organolithium to aryl bromide can be used. Further, advantageously, all three of the reagents (compound of formula (IV), compound of formula (V) and the n-butyllithium) could be present simultaneously in a “one-pot” reaction. The use of n-butyllithium therefore provides the benefits of being safer (i.e. is a milder organolithium reagent), provides increased scalability with improved efficiency (i.e. fewer equivalents of organolithium reagent are required in a simple “one-pot” reaction that can be cooled with ice water rather than dry ice).

The method may additionally comprise the step of synthesising the compound of formula (V), 4-bromophenyl) methoxy-tert-butyl-dimethyl-silane, by protecting 4-bromobenzyl alcohol using tert-butyldimethylsilylchloride and a Lewis base catalyst. The Lewis base catalyst may be imidazole.

As previously indicated, it should be understood that other alcohol protecting groups, other than the TBDMS protecting group, are considered within the scope of the invention. In particular when the protecting group is removable under acid conditions (e.g. removable with HCl in alcohol).

The compound of formula (6) or (6a) may be substantially free of metal ions, optionally substantially free of heavy or transition metal ions, or further optionally substantially free of manganese and/or chromium compounds.

Following oxidation, the compound of formula (6) or (6a) is used to introduce a SiFA moiety to another molecule, for example to form a ligand-SiFA conjugate, for example to form a ligand-SIFA-chelator conjugate

The compound of formula (6) or (6a) may be purified following oxidation. The trituration purification step described herein allows removal of undesired side products such as the compound of formula (7), (7a), (7b) or (7c):

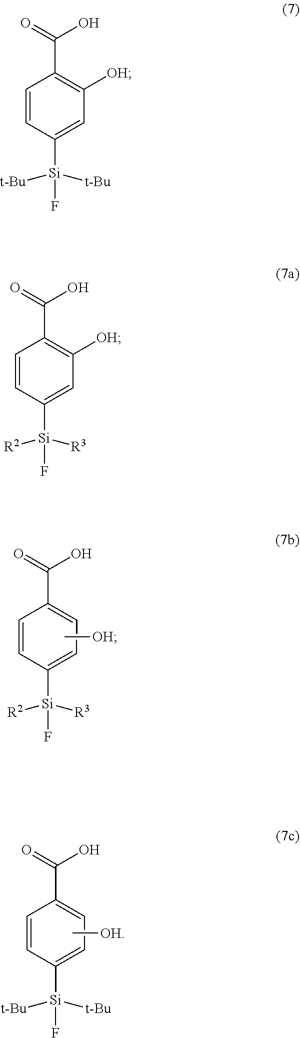

The presence of compounds of formula (7) or (7a) in subsequent reaction steps, for example, may lead to side-products comprising groups of the formula:

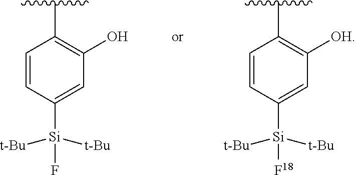

The compound of formula (6) or (6a) may be used in the manufacture of a radiopharmaceutical. The radiopharmaceutical may be a ligand-SIFA-chelator conjugate, comprising:
(a) one or more ligands which are capable of binding to prostate-specific membrane antigen (PSMA);
(b) a silicon-fluoride acceptor (SIFA) moiety which comprises a covalent bond between a silicon and a fluorine atom; and
(c) one or more chelating groups, optionally containing a chelated nonradioactive or radioactive cation.

The radiopharmaceutical may be in the form of a composition, wherein said composition comprises no more than 0.1% (w/w), no more than 0.09% (w/w), no more than 0.08% (w/w), no more than 0.07% (w/w), no more than 0.06% (w/w), no more than 0.05% (w/w), no more than 0.04% (w/w), no more than 0.03% (w/w), no more than 0.02% (w/w) or no more than 0.01% (w/w) of a compound comprising a group of the formula:

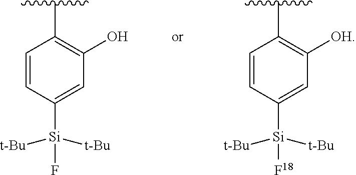

Fluorosilyl compounds with silyl t-butyl groups have been used in PET imaging where the fluorine atoms are enriched with the 18F isotope. The methods herein may thus comprise an additional 18F fluorine exchange step.

The methods herein may additionally comprise the step of reacting a compound of formula (I) a or (6a) with a compound of formula (X) to form a compound of formula (XI)

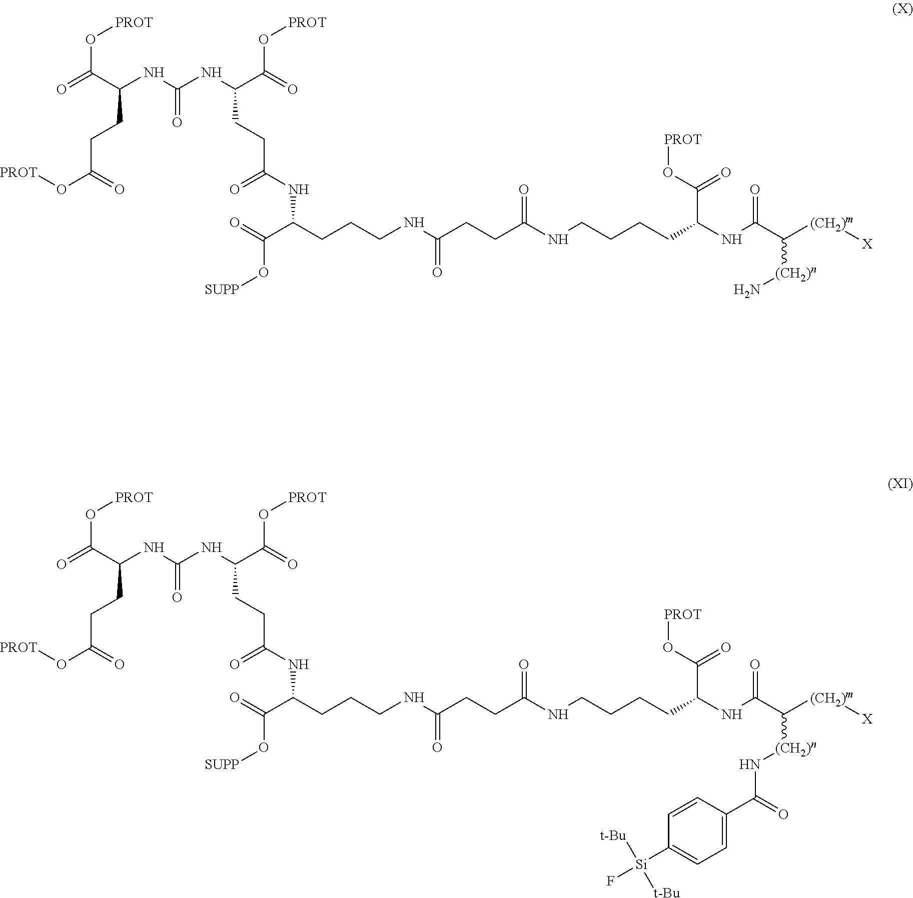

wherein:
SUPP represents a solid support or a link to a solid support;
PROT represents a protecting group;
X represents a protecting group; optionally either a protected amine group or an azide group; and
m and n are independently either 0 or 1.

The compound of formula (XI) may be seen as a PSMA-SiFA conjugate precursor.

The method may further comprise the step of making a compound of formula (XII) from the compound of formula (XI)

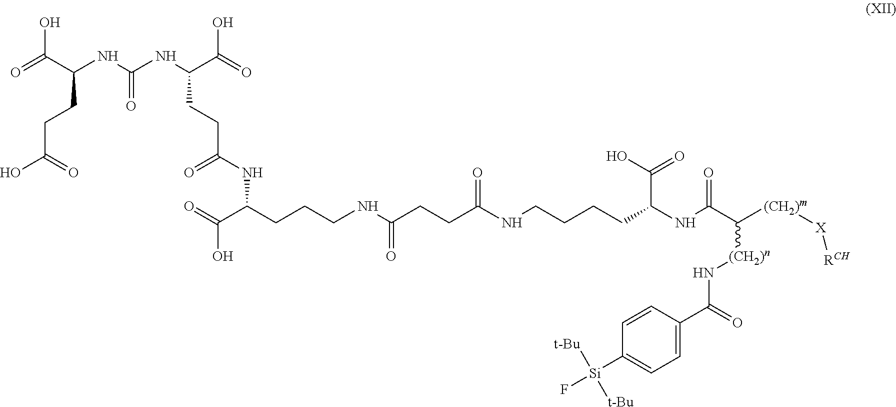

wherein:
m and n are independently either 0 or 1;
R^{CH} is a chelating group;

X is selected from an amide bond, an ether bond, a thioether bond, an ester bond, a thioester bond, a urea bridge, an amine bond, a linking group of the formula

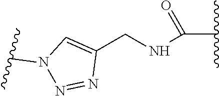 [SMILES: *C(=O)NCc1cn(*)nn1]

wherein the amide bond marked by [symbol] is formed with the chelating group, or a linking group of the formula

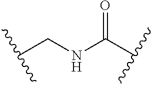 [SMILES: *CNC(*)=O]

wherein the bond marked by [symbol] at the carbonyl end is formed with the chelating group. The compound of formula (XII) may be seen as a PSMA-SiFA conjugate or PSMA-SiFA conjugate precursor with metal ion chelating group R^{CH}.

The chelating group of the ligand-SIFA-chelator conjugate or R^{CH} may be selected from: bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), N′-[5-[acetyl(hydroxy)amino]pentyl]-N-[5-[4-[5-aminopentyl-(hydroxy)amino]-4-oxobutanoyl]amino]pentyl]-N-hydroxybutandiamide (DFO), 4,11-bis(carboxymethyl)-1,4,8,11-tetraazabicyclo[6.6.2]hexadecan (DO2A) 1,4,7,10-tetracyclododecan-N,N′,N″,N′-tetraacetic acid (DOTA), α-(2-carboxyethyl)-1,4,7,10-tetraazacyclododecane-1,4,7,10-tetraacetic acid (DOTAGA), 1,4,7,10 tetraazacyclododecane N,N′,N″,N″ 1,4,7,10-tetra(methylene)phosphonic acid (DOTMP), N,N′-dipyridoxylethylendiamine-N,N′-diacetate-5,5′-bis(phosphat) (DPDP), diethylene triamine N,N′,N″ penta(methylene)phosphonic acid (DTMP), diethylenetriaminepentaacetic acid (DTPA), ethylenediamine-N,N′-tetraacetic acid (EDTA), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), tetra 3-hydroxy-N-methyl-2-pyridinone chelators (4-((4-(3-(bis(2-(3-hydroxy-1-methyl-2-oxo-1,2-dihydropyridine-4-carboxamido)ethyl)amino)-2-((bis(2-(3-hydroxy-1-methyl-2-oxo-1,2-dihydropyridine-4-carboxamido)ethyl)amino)methyl) propyl)phenyl)amino)-4-oxobutanoic acid), abbreviated as Me-3,2-HOPO, 1,4,7-triazacyclononan-1-succinic acid-4,7-diacetic acid (NODASA), 1-(1-carboxy-3-carboxypropyl)-4,7-(carbooxy)-1,4,7-triazacyclononane (NODAGA), 1,4,7-triazacyclononanetriacetic acid (NOTA), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), tris(hydroxypyridinone) (THP), terpyridin-bis(methyleneamintetraacetic acid (TMT), 1,4,7-triazacyclononane-1,4,7-tris[methylene (2-carboxyethyl)phosphinic acid](TRAP), 1,4,7,10-tetraazacyclotridecan-N,N′,N″,N″-tetraacetic acid (TRITA), 3-[4,7-bis [[2-carboxyethyl(hydroxy)phosphoryl]methyl]-1,4,7-triazonan-1-yl]methyl-hydroxy-phosphoryl]propanoic acid, and triethylenetetraaminehexaacetic acid (TTHA).

The chelating group of the ligand-SIFA-chelator conjugate or R^{CH}may be selected from: 1,4,7,10-tetracyclododecan-N,N′,N″,N′-tetraacetic acid (DOTA), α-(2-carboxyethyl)-1,4,7,10-tetraazacyclododecane-1,4,7,10-tetraacetic acid (DOTAGA) or 1,4,7-triazacyclononane-1,4,7-tris[methylene (2-carboxyethyl)phosphinic acid](TRAP).

The chelating group of the ligand-SIFA-chelator conjugate or R^{CH} may be selected from:

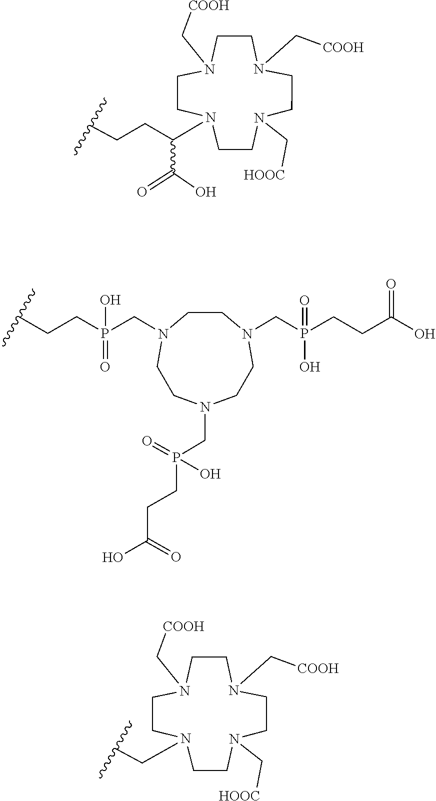 [SMILES: *CCC(C(=O)O)N1CCN(CC(=O)O)CCN(CC(=O)O)CCN(CC(=O)O)CC1 ; *CCP(=O)(O)CN1CCN(CP(=O)(O)CCC(=O)O)CCN(CP(=O)(O)CCC(=O)O)CC1 ; *CN1CCN(CC(=O)O)CCN(CC(=O)O)CCN(CC(=O)O)CC1]

The chelating group may be tailored to suit need, so as to beneficially hold an ion, such as a radioisotopic metal.

The chelating group may comprise a chelated cation which may be radioactive or non-radioactive. The chelating group may comprise a chelated metal cation which may be radioactive or non-radioactive. Examples of cations that may be chelated by the chelating group are the cations of ⁴³Sc, ⁴⁴Sc, ⁴⁷Sc, ⁵¹Cr, ^{52m}Min, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁷Ga, ⁶⁸Ga, ⁸⁹Zr, ⁹⁰Y, ⁸⁹Y, ^{99m}Tc, ⁹⁷Ru, ¹⁰⁵Rh, ¹⁰⁹Pd, ¹¹¹Ag, ^{110m}In, ¹¹¹In, ^{113m}In, ^{114m}In, ^{117m}Sn, ¹²¹Sn, ¹²⁷Te, ¹⁴²Pr, ¹⁴³Pr, ¹⁴⁹Pm, ¹⁵¹Pm, ¹⁴⁹Tb, ¹⁵²Tb, ¹⁵⁵Tb, ¹⁶¹Tb, ¹⁵³Sm, ¹⁵⁷Gd, ¹⁶¹Tb, ¹⁶⁶Ho, ¹⁶⁵Dy, ¹⁶⁹Er, ¹⁶⁹Yb, ¹⁷⁵Yb, ¹⁷²Tm, ¹⁷⁷Lu, ¹⁸⁶Re, ¹⁸⁸Re, ¹⁹¹Pt, ¹⁹⁷Hg, ¹⁹⁸Au, ¹⁹⁹Au, ²¹²Pb, ²⁰³Pb, ²¹¹At., ²¹²Bi, ²¹³Bi, ²²³Ra, ²²⁵Ac, ²²⁷Th, a cationic molecule comprising ¹⁸F or a cation such as ¹⁸F-[AIF]²⁺. The chelated cation may be a radioactive cation selected from the cations of Sc, Cu, Ga, Y, In, Tb, Ho, Lu, Re, Pb, Bi, Ac, Er and Th. The chelated cation may be a non-radioactive cation selected from the cations of Sc, Cu, Ga, Y, In, Tb, Ho, Lu, Re, Pb, Bi, Ac, Th and Er. The cation may be Ga. The cation may be Lu. The chelated cation may be a nonradioactive Ga³⁺ cation.

The compound of formula (XII) may be selected from:

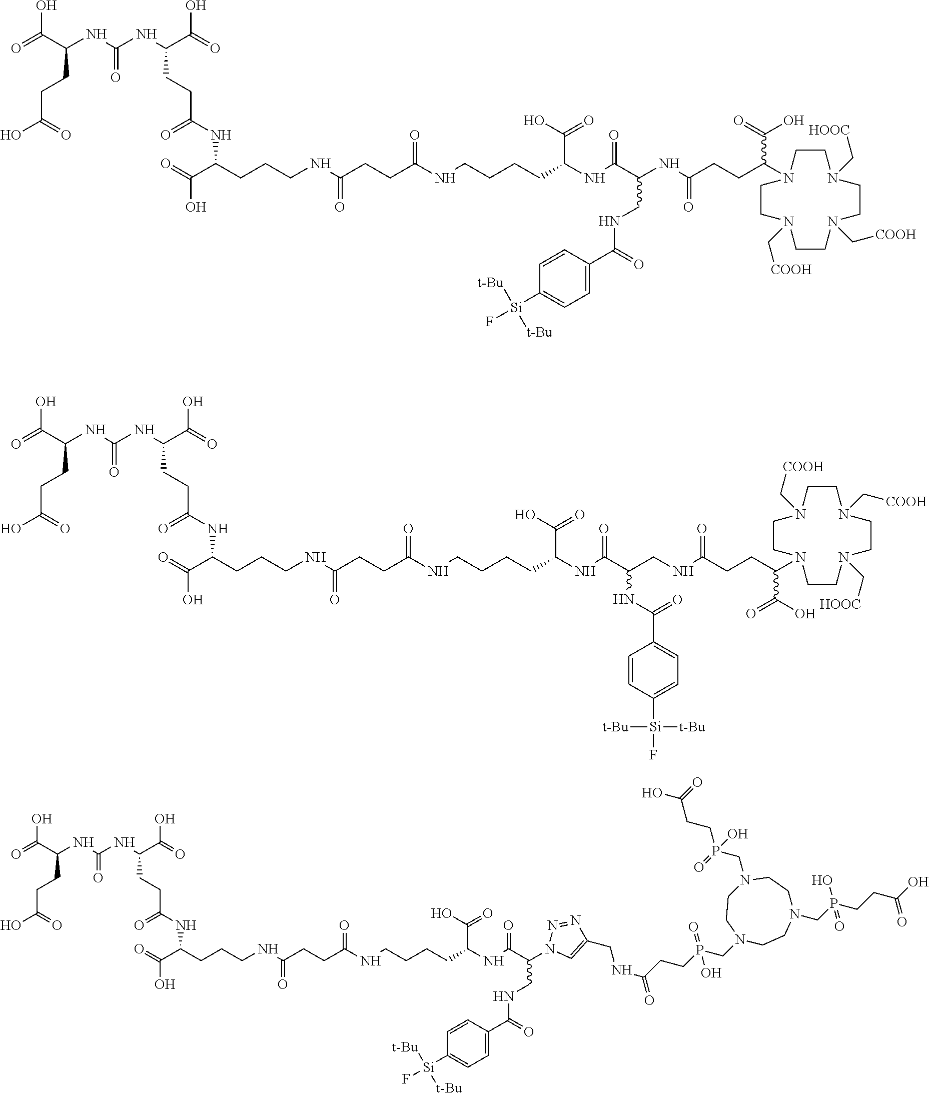

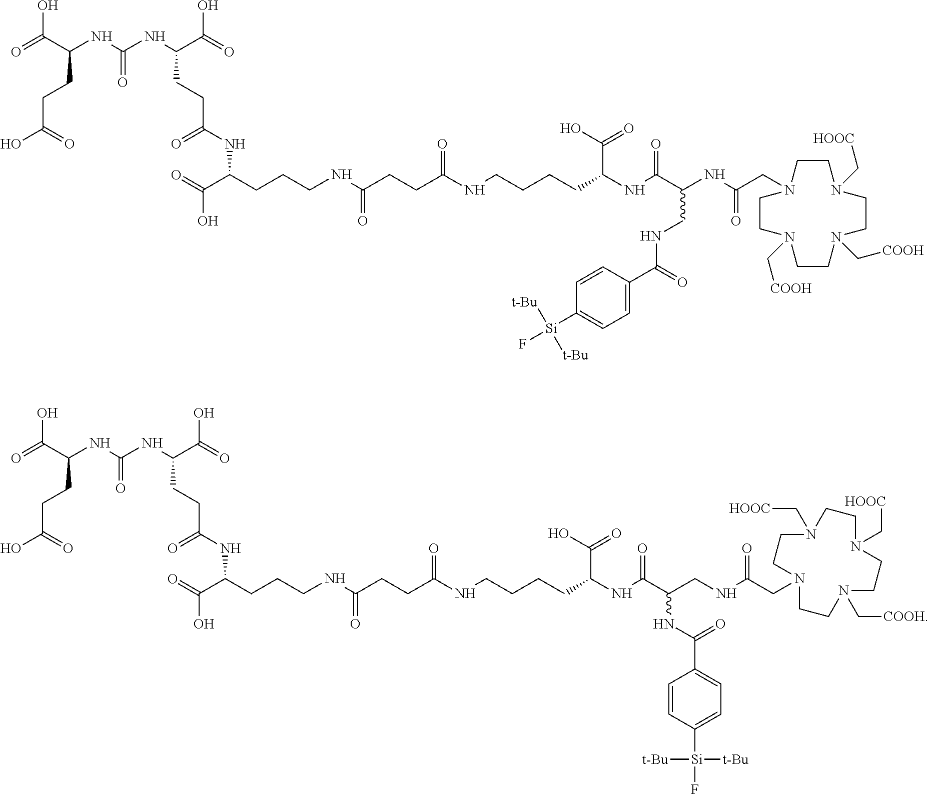 [SMILES: CC(C)(C)[Si](F)(c1ccc(C(=O)NCC(NC(=O)CN2CCN(CC(=O)O)CCN(CC(=O)O)CCN(CC(=O)O)CC2)C(=O)N[C@H](CCCCNC(=O)CCC(=O)NCCC[C@@H](NC(=O)CC[C@H](NC(=O)N[C@@H](CCC(=O)O)C(=O)O)C(=O)O)C(=O)O)C(=O)O)cc1)C(C)(C)C ; CC(C)(C)[Si](F)(c1ccc(C(=O)NC(CNC(=O)CN2CCN(CC(=O)O)CCN(CC(=O)O)CCN(CC(=O)O)CC2)C(=O)N[C@H](CCCCNC(=O)CCC(=O)NCCC[C@@H](NC(=O)CC[C@H](NC(=O)N[C@@H](CCC(=O)O)C(=O)O)C(=O)O)C(=O)O)C(=O)O)cc1)C(C)(C)C]

The compound of formula (XII) may be a compound of formula (XIII)

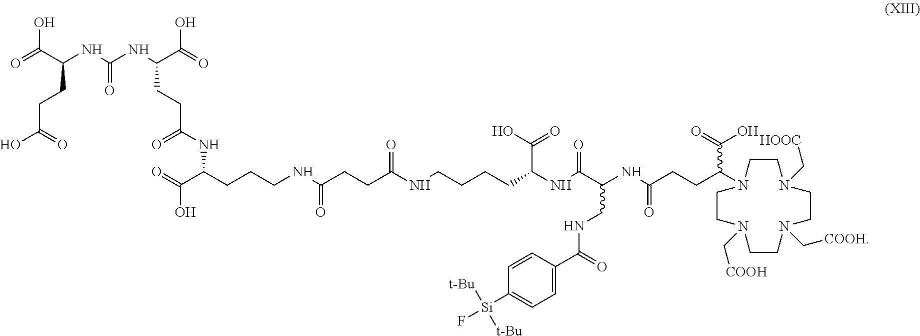 [SMILES: CC(C)(C)[Si](F)(c1ccc(C(=O)NCC(NC(=O)CCC(C(=O)O)N2CCN(CC(=O)O)CCN(CC(=O)O)CCN(CC(=O)O)CC2)C(=O)N[C@H](CCCCNC(=O)CCC(=O)NCCC[C@@H](NC(=O)CC[C@H](NC(=O)N[C@@H](CCC(=O)O)C(=O)O)C(=O)O)C(=O)O)C(=O)O)cc1)C(C)(C)C]

The compound of formula (XIII) may be a compound of formula (XIV)

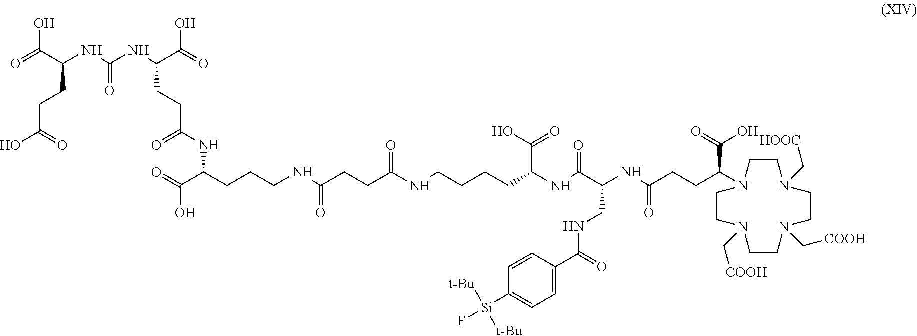 [SMILES: CC(C)(C)[Si](F)(c1ccc(C(=O)NC[C@@H](NC(=O)CC[C@@H](C(=O)O)N2CCN(CC(=O)O)CCN(CC(=O)O)CCN(CC(=O)O)CC2)C(=O)N[C@H](CCCCNC(=O)CCC(=O)NCCC[C@@H](NC(=O)CC[C@H](NC(=O)N[C@@H](CCC(=O)O)C(=O)O)C(=O)O)C(=O)O)C(=O)O)cc1)C(C)(C)C]

The method may additionally comprise enriching the fluorine atoms of a compound of any of formulae (I) to (IV) and (X) to (XIII) or compound (XIV) with the fluorine-18 isotope. For example, the radio isotope of fluorine may be introduced as the result of an isotope exchange reaction, where for example a compound of formula (I) or compound (XIV) is enriched with the fluorine-18 isotope using [18F-]/Kryptofix 2.2.2/K+ as substantially described in Angew Chem Int Ed Engl. 2006 Sep. 11;45 (36): 6047-50, or in a manner known to the person skilled in the art. The reaction may be carried out in a dipolar aprotic solvent.

The method may additionally comprise enriching the fluorine atoms of a compound of any of formulae (XI) to (XIII) or compound (XIV) with the fluorine-18 isotope. The method may comprise enriching the fluorine atoms of a compound of formulae (XI) to (XIII) or compound (XIV) to over 50%, 60%, 70%, 80%, 85%, 90%, 95% or 98% with the fluorine-18 isotope. The fluorine-18 isotope is beneficially useful in at PET imaging.

The method may comprise chelating a radioactive metal cation in the chelating group; alternatively, the method may comprise chelating a non-radioactive metal cation in the chelating group. The chelating group may be chelating a radioactive cation and the chelated radioactive cation may be selected from 68Ga, 177Lu or 225Ac. Beneficially, the radioactive metal ions are useful in imaging and/or radio treatment. The chelating group may be chelating a non-radioactive cation and the chelated non-radioactive cation may be natGa.

In the methods herein, the ligand-SIFA-chelator conjugate may be selected from:

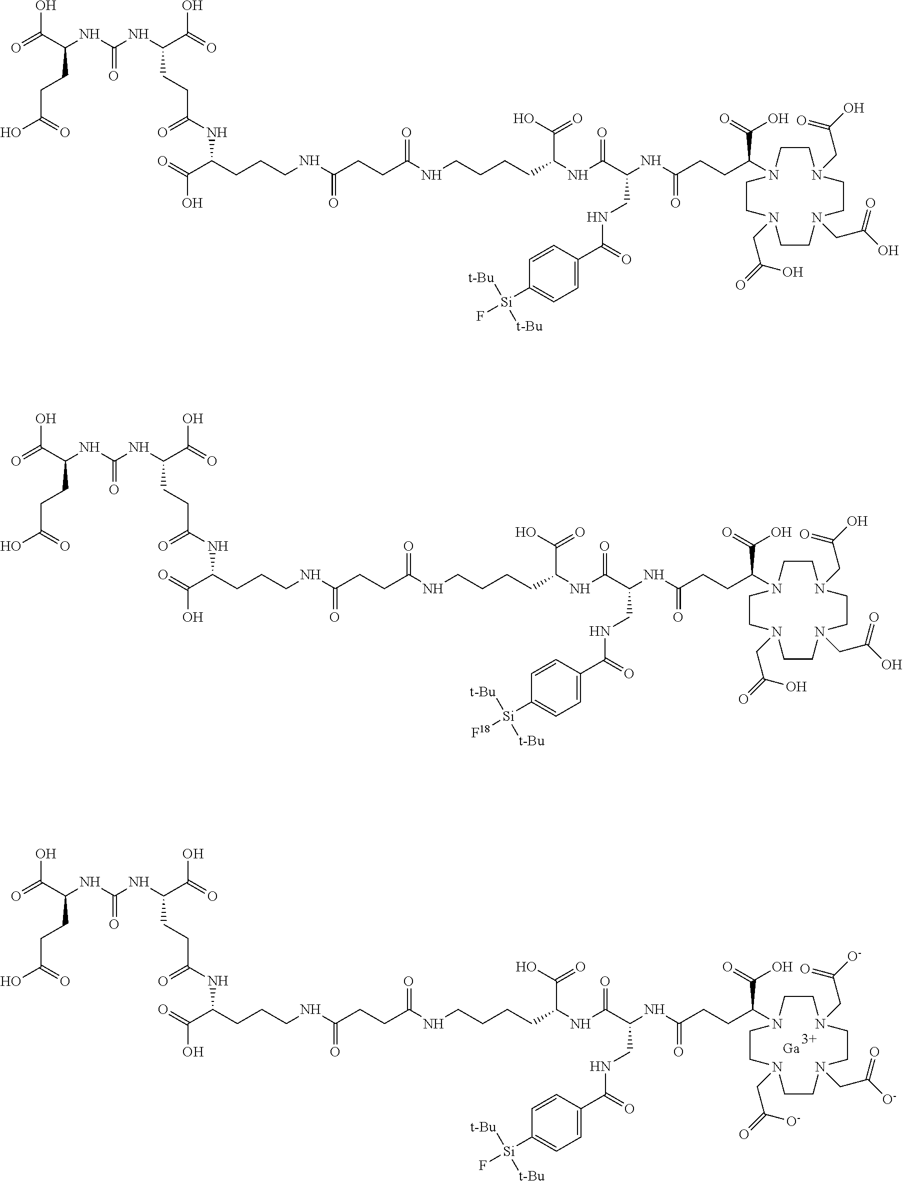

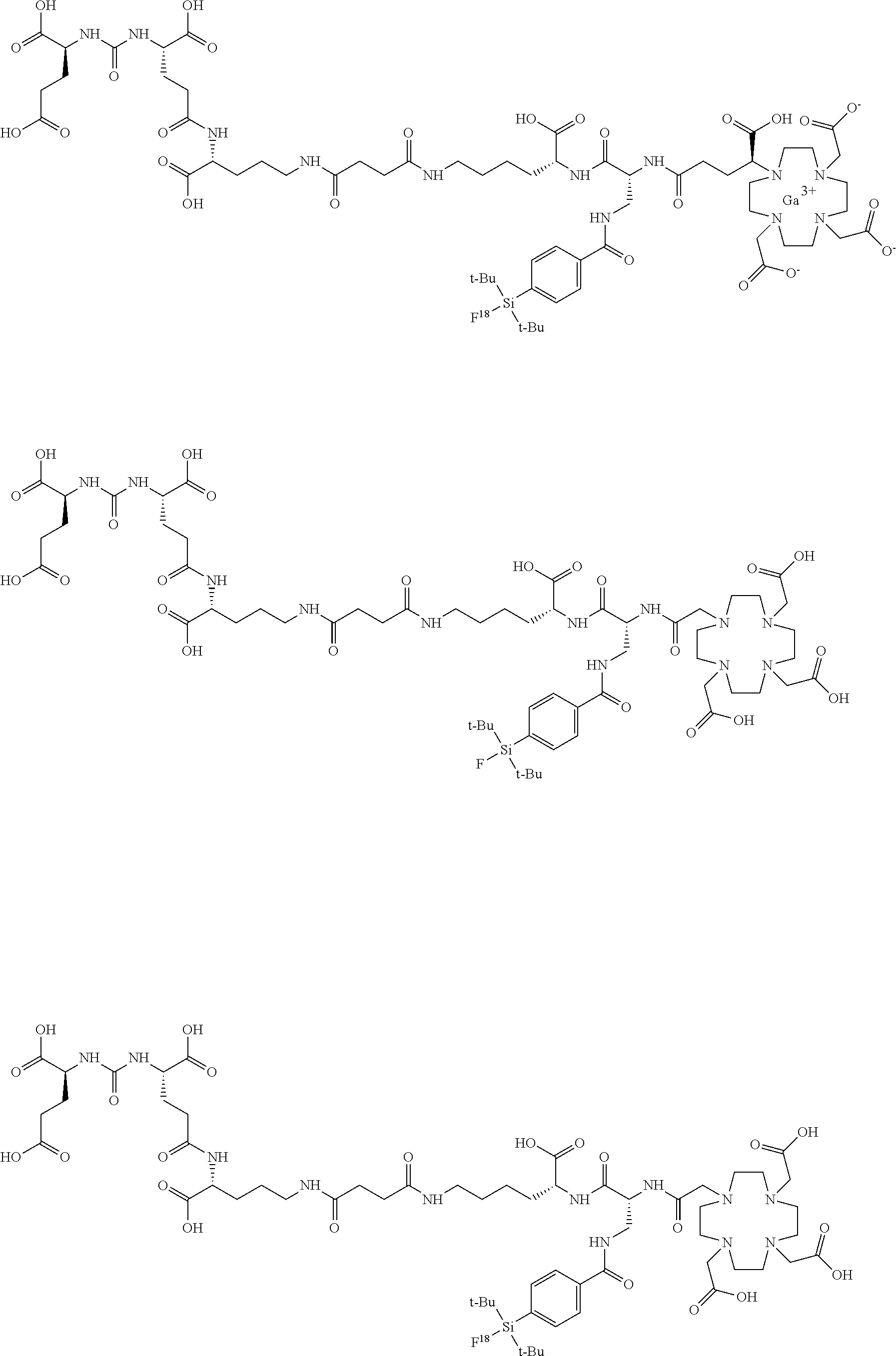

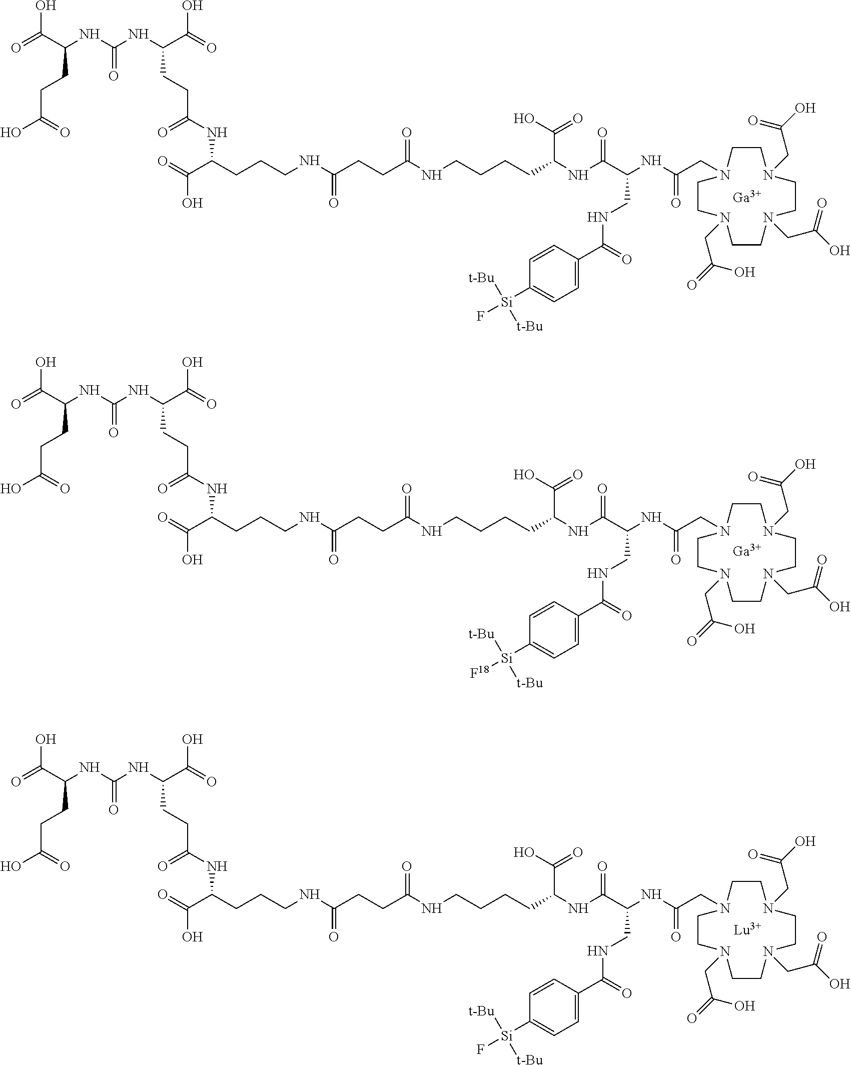

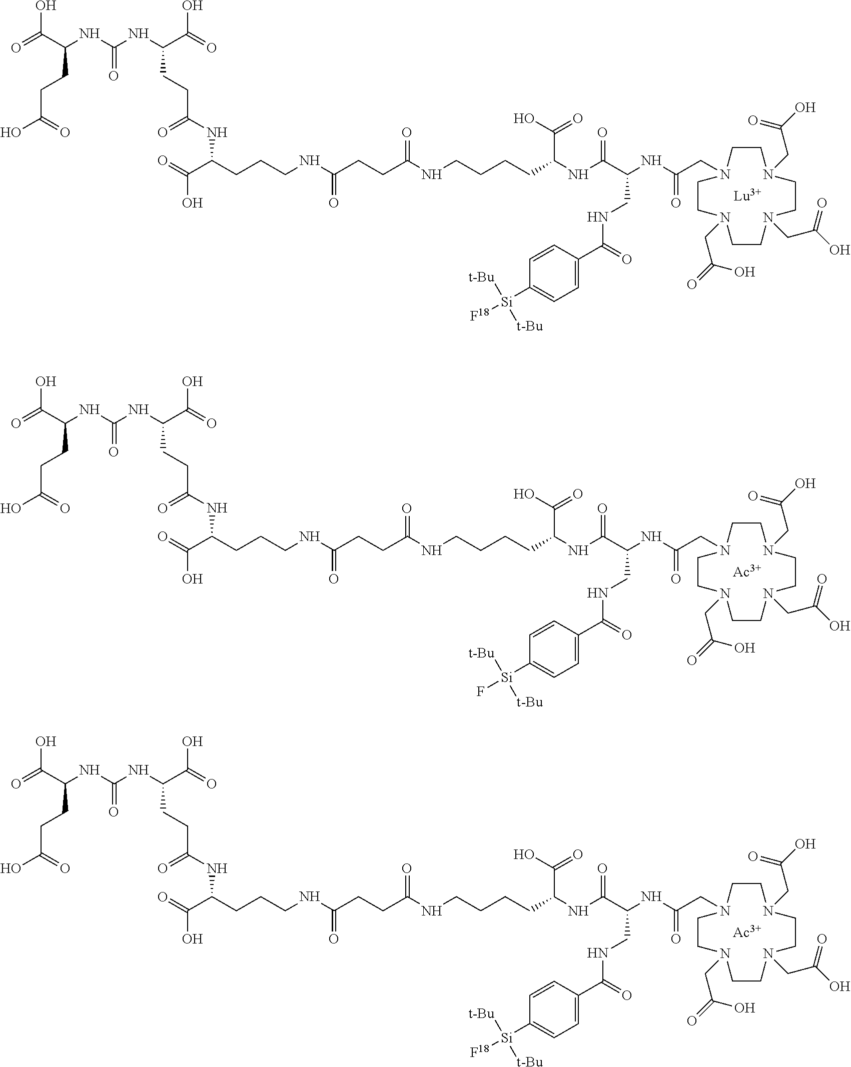

Also provided is a composition comprising a ligand-SIFA-chelator conjugate selected from:

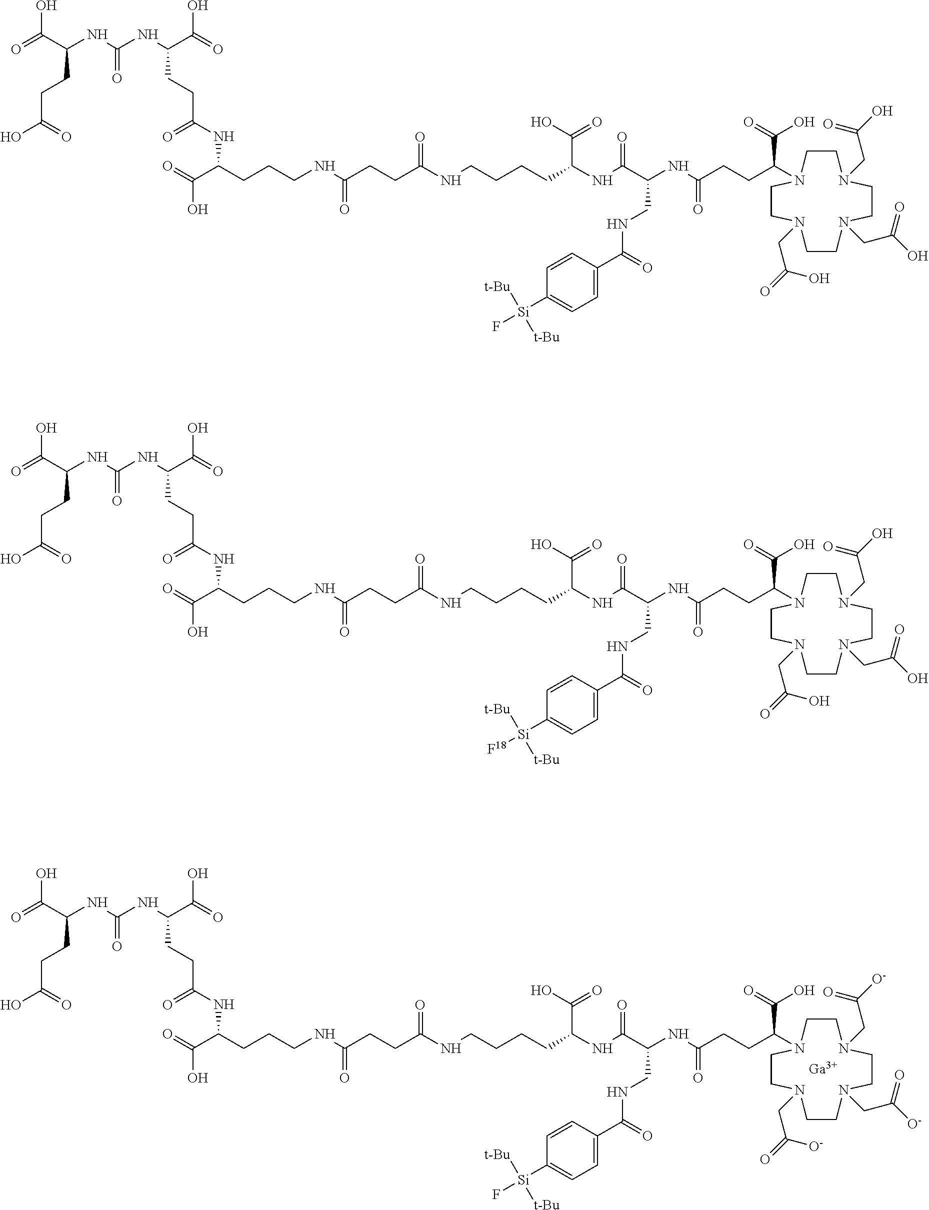

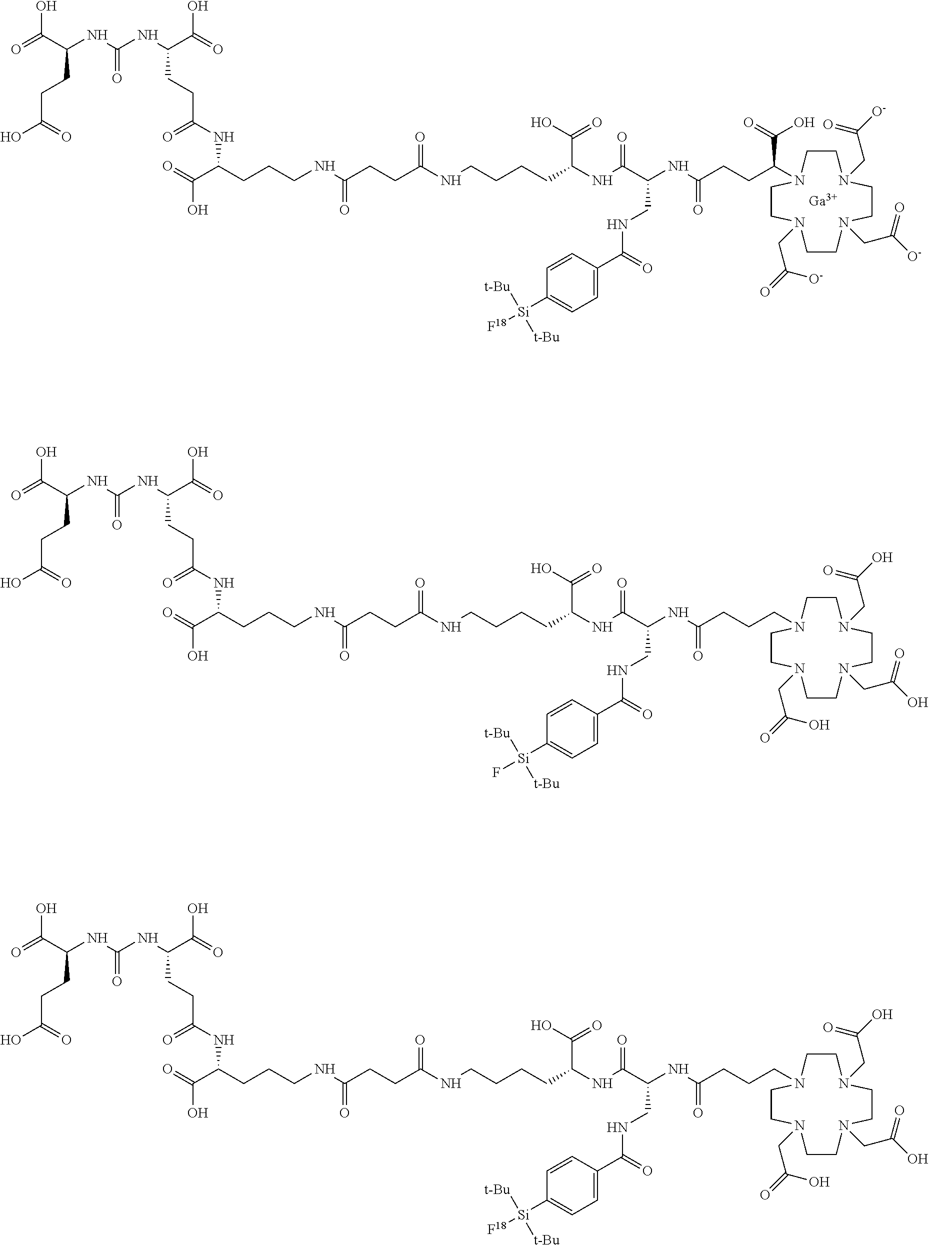

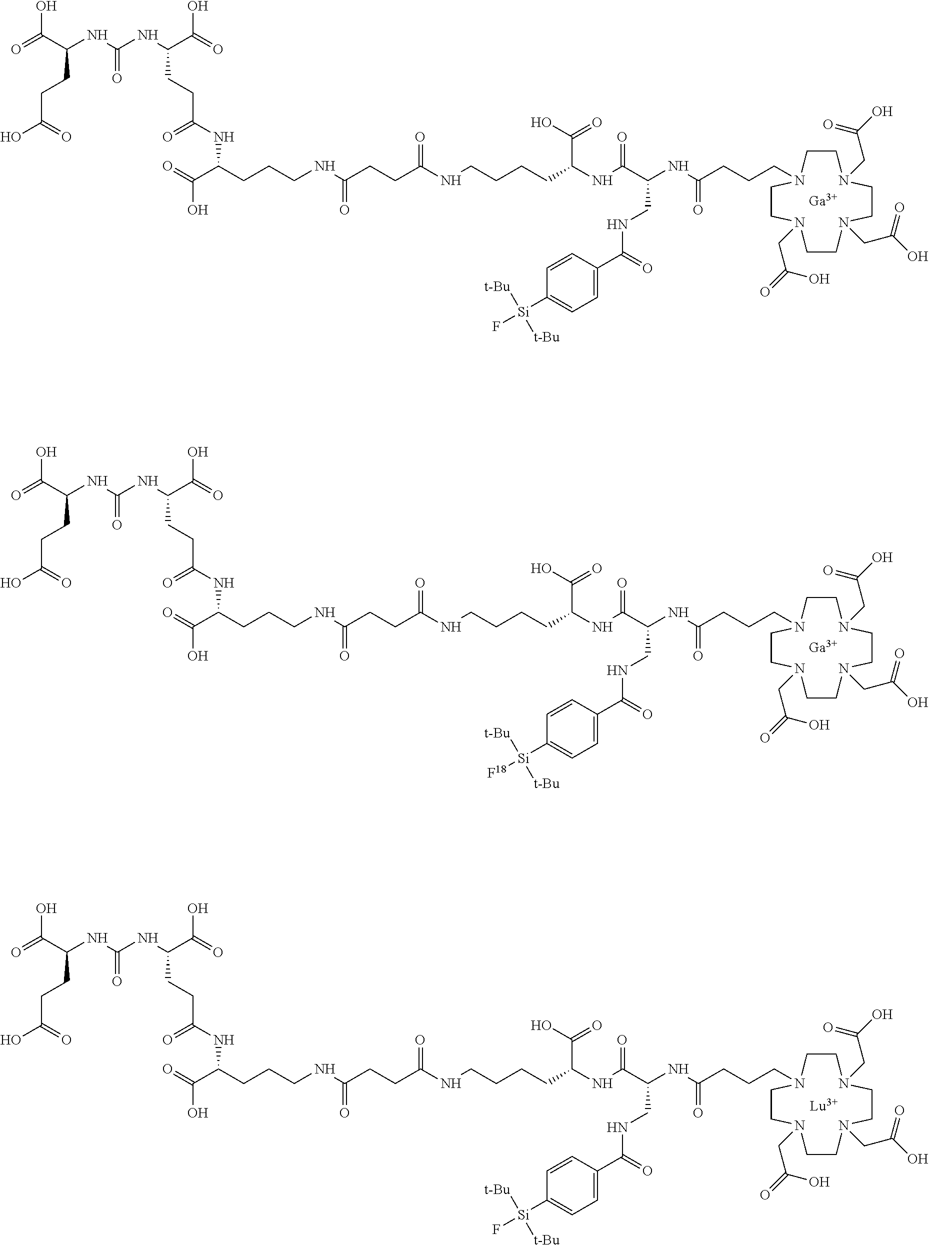

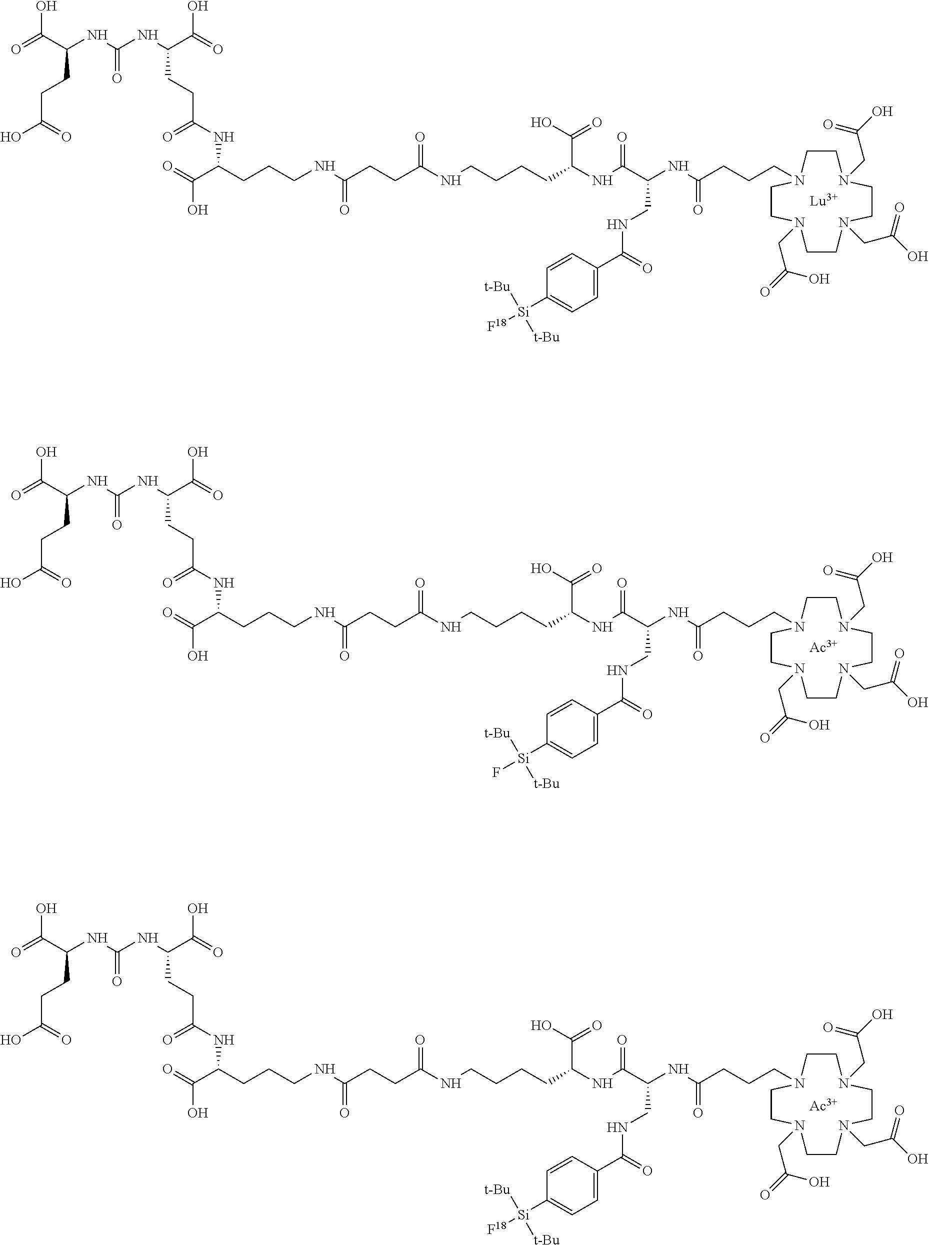

wherein said compound is prepared using a method as described herein, and wherein said composition comprises no more than 0.1% (w/w), no more than 0.09% (w/w), no more than 0.08% (w/w), no more than 0.07% (w/w), no more than 0.06% (w/w), no more than 0.05% (w/w), no more than 0.04% (w/w), no more than 0.03% (w/w), no more than 0.02% (w/w) or no more than 0.01% (w/w) of a compound comprising a group of the formula:

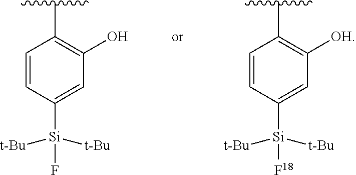

The method may additionally comprise the step of using a ligand-SIFA-chelator conjugate or a compound of formulae (XII or XIII), compound (XIV), or compounds further derived therefrom in the diagnosis and/or treatment of disease. The method may additionally comprise the step of using a ligand-SIFA-chelator conjugate or a compound of formulae (XII) or (XIII), compound (XIV), or compounds further derived therefrom in the diagnosis of disease, wherein any fluorine atom is enriched with the fluorine-18 isotope. It may be understood that when administered, e.g. to the human or animal body, the ligand-SIFA-chelator conjugate or compound of formulae (XII or XIII) can preferentially bind to the area of interest, and then for example the isotopes present in the ligand-SIFA-chelator conjugate or compounds of formulae (XII or XIII) may be useful in imaging the area the compound has bound to. This may be useful in diagnosis and/or in deciding on a method of treatment.

The method may additionally comprise the step of using a ligand-SIFA-chelator conjugate or a compound of formulae (XII) or (XIII), compound (XIV), or compounds further derived therefrom in the diagnosis and/or treatment of disease, wherein the chelating group is chelating a radioactive cation, wherein optionally the radioactive cation may be selected from 68Ga, 177Lu, or 225Ac. The method may additionally comprise the step of using a ligand-SIFA-chelator conjugate or a compound of formulae (XII) or (XIII), compound (XIV), or compounds further derived therefrom in the diagnosis and/or treatment of disease, wherein the chelating group is chelating a non-radioactive cation, wherein optionally the non-radioactive cation may be natGa.

It may be understood that when administered, e.g. to the human or animal body, the ligand-SIFA-chelator conjugate or compound of formulae (XII) or (XIII) or compound (XIV) can preferentially bind to the area of interest. Then for example the isotopes present in the ligand-SIFA-chelator conjugate or compound of formulae (XII) or (XIII) or compound (XIV) may be useful in treating the area the compound has bound to. For example, this may be useful in bringing radioisotopes proximate to diseased cells/tissue.

Herein disclosed, is the use of any one of the methods disclosed herein to make the corresponding compounds disclosed herein, and/or any derivative moieties made therefrom. By way of non-limiting example, herein disclosed is the use of the method disclosed in the first aspect of the invention to make the compound of formula (I), (I) a or (6a).

Herein disclosed, are the compounds disclosed herein when made by the corresponding methods disclosed herein, and/or any derivative moieties made therefrom. By way of non-limiting example, herein disclosed is the compound of formula (I) (I) a or (6a) when made using the method disclosed in the first aspect of the invention.

The compound of formula (I) a/(6a) may in particular be prepared in accordance with the following scheme:

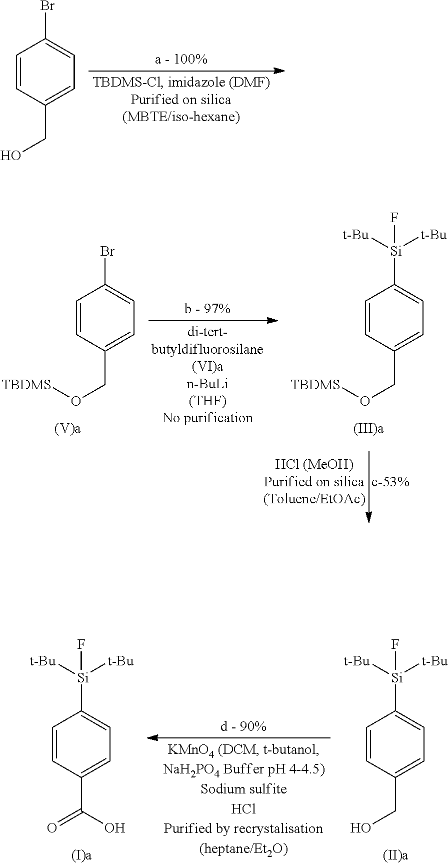

Step a—the alcohol group of 4-bromobenzyl alcohol was protected with a TBDMS group to give 4-bromophenyl) methoxy-tert-butyl-dimethyl-silane (corresponding to the compound of formula (2d) or an embodiment of the compound of formula (V) wherein TBDMS is used to protect the alcohol herein termed compound (V) a).

Step b—the bromine group of the (4-bromophenyl) methoxy-tert-butyl-dimethyl-silane (compound (V) a) was exchanged for a di-alkylfluorosilyl group by reacting it with tert-butyl-difluorosilane (corresponding to the compound of formula (4a) or an embodiment of the compound of formula (IV) wherein the R groups are tert-butyl groups herein termed compound (IV) a) and n-butyllithium to give di-tert-butyl-[4-[[tert-butyl(dimethyl)silyl]oxymethyl]phenyl]-fluoro-silane (corresponding to the compound of formula (Ib) or an embodiment of the compound of formula (III) wherein the R groups are tert-butyl groups herein termed compound (III) a).

Step c—the TBDMS protecting group of compound (III) a was removed using acid and the resultant compound, 4-(di-tert-butyl(fluoro)silyl)benzyl alcohol (corresponding to the compound of formula (5a) or an embodiment of the compound of formula (II) wherein the R groups are tert-butyl groups herein termed compound (II) a) was purified by column chromatography.

Step d—the alcohol group of 4-(di-tert-butyl(fluoro)silyl)benzyl alcohol (compound (II) a) was oxidised to the corresponding benzoic acid to give 4-(di-tert-butyl(fluoro)silyl)benzoic acid (corresponding to the compound of formula (6a) or an embodiment of the compound of formula (I); compound (I) a).

The below experimental is provided for the synthesis of 4-(di-tert-butyl(fluoro)silyl)benzoic acid (compound (I) a), corresponding to the synthesis of the compound of formula (6a) or an embodiment of the compound of formula (I).

### Example A—Preparation of 4-(di-tert-butyl(fluoro)silyl)benzoic acid

Step a—Synthesis of (4-bromophenyl) methoxy-tert-butyl-dimethyl-silane (V) a

To a solution of 4-bromobenzyl alcohol (113.5 g, 606.8 mmol, 1.0 eq.) in DMF (1100 mL) was added imidazole (49.57 g, 728 mmol, 1 eq.) and TBDMS-Cl (98.78 g, 655.38 mmol, 1.08 eq). The mixture was stirred at room temperature overnight.

The reaction mixture was diluted with water (1100 mL) and extracted with MTBE (2×500 ml then 2×300 mL). The combined organics were dried over MgSO₄, filtered and evaporated to a crude oil. The material was purified in two batches on silica eluting with 2% MTBE/iso-hexane to give the desired material as a colourless oil (184.0 g, 100%).

Step b—Synthesis of di-tert-butyl-[4-[[tert-butyl(dimethyl)silyl]oxymethyl]phenyl]-fluoro-silane (III) a

To a solution of (4-bromophenyl) methoxy-tert-butyl-dimethyl-silane ((V) a; 17.85 g, 59.23 mmol) and tert-butyl-difluorosilane (12.28 g, 68.11 mmol) in THF (170 mL) cooled to +1° C. (internal) was added n-butyllithium solution (2.5M, 27.24 mL, 68.11 mmol) dropwise over 1 hr. The solution was stirred at +1° C. (internal) over 2 hrs then allowed to warm to +15° C. (internal) over 30 mins. The solution was diluted with MTBE (200 mL) and washed with sat. brine (200 mL). The aqueous was diluted with water (50 mL) and extracted into MTBE (3×200 mL). The combined organics were dried over MgSO₄, filtered and evaporated to a yellow oil (21.89 g, 97%). Material used without purification.

Step c—Synthesis of 4-(di-tert-butyl(fluoro)silyl)benzyl alcohol (II) a

To a solution of di-tert-butyl-[4-[tert-butyl(dimethyl)silyl]oxymethyl]phenyl]-fluoro-silane ((III) a; 21.89 g, 57.20 mmol) in methanol (700 mL) was added conc. HCl (11 mL) dropwise with stirring over 10 mins. The solution was then stirred at room temperature overnight.

The reaction mixture was evaporated to remove volatile organics and the residue was treated with sat. aq. Sodium bicarbonate (120 mL) until pH8. The mixture was extracted into MTBE (3×200 mL). The combined organics were dried over MgSO₄, filtered and evaporated to a pale yellow oil.

The oil was diluted with toluene (10 mL) and loaded onto a 330 g pre-wetted (toluene)silica column. The column was eluted with a gradient of 0-10% ethyl acetate in toluene over 10 column volumes. Fractions were analysed by TLC (5% ethyl acetate/toluene, stained with KMnO₄) with only the purest fractions retained and combined to give the desired material as a white solid (8.078 g, 53%). Some mixed fractions were retained and combined with those from other batches for re-purification.

Step d—Synthesis of 4-(di-tert-butyl(fluoro)silyl)benzoic acid (I) a

To a solution of 4-(di-tert-butyl(fluoro)silyl)benzyl alcohol ((II) a; 33.17 g, 123.58 mmol) in tert-butanol (370 mL) and DCM (75 mL) was added an aqueous solution of KMnO₄ (29.3 g in 185 mL) as a thin stream over 5 mins. This was followed by a solution of NaH₂PO₄.HCl in water (56.27 g in 200 mL) as a thin stream over 10 mins giving a rise in internal temperature from +20° C. to +35° C. The mixture was stirred over 2 hrs then an aliquot was removed, treated as per the workup procedure, isolated and analysed by NMR to show the reaction was complete.

The reaction was quenched by the addition of sat. aq. NaSO3 (700 mL) and stirred over 30 mins. c. HCl (110 mL) was added dropwise over 10 mins then stirred over 30 mins. The now white cloudy mixture extracted into MTBE (1000 ml then 2×500 mL). The combined extracts dried over MgSO₄, filtered and evaporated to a white solid (34.7 g, 99%).

This material was combined with two other batches (combined weight 111.06 g), dissolved in MTBE (1L) at 50° C. and then evaporated to a white solid (114.06 g) with some residual MTBE. The solid was suspended in 25:1 heptane: Et₂O (720 mL) and stirred at 65° C. over 30 mins. The mixture was allowed to cool to r.t. with stirring then cooled to 0° C. with ice over 20 mins. The mixture was filtered and the filter cake washed with ice-cold 25:1 heptane: Et₂O (3×100 mL) followed by n-pentane (500 mL) The resulting white solid was dried under vacuum at room temperature to constant weight. Yield 102.28 g (90% of theoretical yield).

### Example B—Invention Versus Comparative Reaction Conditions for Step b

A summary of comparative reaction conditions for step b (synthesis of di-tert-butyl-[4-[tert-butyl(dimethyl)silyl]oxymethyl]phenyl]-fluoro-silane) is shown in Table 1.

Table 1, Entry 1 represents literature conditions (i.e. step-wise addition of alkyllithium reagent to 4-(TBS-oxymethyl) bromobenzene using tert-BuLi at cryogenic temperatures, followed by addition of t-Bu₂SiF₂). These conditions resulted in large quantities of side-product formation and due to the requirement for cryogenic temperatures and the use of tert-BuLi. are considered to be unsuitable for scale up.

Entry 2 represents use of n-BuLi instead of tert-BuLi in the procedure of Entry 1, without substantive modification of reaction conditions (i.e. step-wise addition and cryogenic temperatures were still used). This resulted in a mixture of products with the desired product being obtained in a lower yield.

Entry 3 represents a procedure according to the present invention, wherein n-BuLi is added to a mixture comprising 4-(TBS-oxymethyl) bromobenzene and t-Bu₂SiF₂ (i.e. one-pot). This resulted in an improved yield of desired product compared to the procedures of Entry 1 and 2, without requiring tert-BuLi or cryogenic temperatures.

Entry 4 represents use of tert-BuLi instead of n-BuLi in the procedure of Entry 3, without substantive modification of reaction conditions (i.e. one-pot and non-cryogenic temperatures). This resulted in a mixture of products with the desired product being obtained in poor yield.

Entries 5 and 6 represent procedures according to the present invention. Feasibility at larger scales is demonstrated.

TABLE 1
Invention versus comparative reaction conditions for step b (Example B)

| Entry | Scale | | Conditions | | RM (eq.) | Purification | Results |
|---|---|---|---|---|---|---|---|
| 1 | 500 | mg | 1) | 4-(TBS- | tert-BuLi | Aqueous | Yield = |
| | | | | oxymethyl)bromobenzene | (1.55M/pentanes; | work-up | 60-70% |
| | | | | (1.0 eq), THF, −78° C. | 2.1 eq.) | | (conversion |
| | | | | Add RM and stir at −78° | | | by NMR) |
| | | | | C. for 8 minutes. | | | |
| | | | 2) | Add tBu2SiF₂ rapidly (1.2 | | | |
| | | | | eq.), THF, −78° C. | | | |
| | | | 1) | 2 min at −78° C. then remove | | | |
| | | | | from cold bath | | | |
| 2 | 520 | mg | 2) | 4-(TBS- | n-BuLi dropwise | Aqueous | Yield = ~40% |
| | | | | oxymethyl)bromobenzene | over 2 min. | work-up | (conversion by |
| | | | | (1.0 eq), THF, −78° | (2.5M/hexanes; | | NMR) |
| | | | | C. Add RM and stir at −78° | 1.2 eq.) | | |
| | | | | C. for 20 minutes. | | | |
| | | | 3) | Add tBu₂SiF₂ (1.2 eq.), | | | |
| | | | | THF, −78° C. | | | |
| | | | 3) | 30 min at −78° C. then allow to | | | |
| | | | | warm to 0° C. over 2 h | | | |
| 3 | 250 | mg | 1. | 4-(TBS- | n-BuLi dropwise | Aqueous | Yield = 75% |
| | | | | oxymethyl)bromobenzene | over 2 min | work-up | (conversion by |
| | | | | (1.0 eq), tBu₂SiF₂ (1.2 eq.), | maintaining temp | | NMR) |
| | | | | THF, 0-5° C. Add RM. | below 5° C. | | |
| | | | 2. | 60 min at 0° C. | (2.5M/hexanes; | | |
| | | | | | 1.2 eq.) | | |
| 4 | 150 | mg | 1. | 4-(TBS- | t-BuLi | Aqueous | Yield = N/A |
| | | | | oxymethyl)bromobenzene | (1.7M in | work-up | |
| | | | | (1.0 eq), tBu₂SiF₂ (1.2 eq.), | pentanes) | | |
| | | | | THF, 0° C. Add RM. | (titrated to 1.55M, | | |
| | | | 2. | 60 min at 0° C. | 1.2 eq.) | | |
| 5 | 2.5 | g | 1. | 4-(TBS- | n-BuLi dropwise | Aqueous | Yield = 75% |
| | | | | oxymethyl)bromobenzene | over 5 min | work-up | (conversion by |
| | | | | (1.0 eq), tBu₂SiF₂ (1.15 eq.), | (2.5M/hexanes; | | NMR) |
| | | | | THF, 0-5° C. Add RM. | 1.15 eq.) | | |
| | | | 2. | 60 min at 0-5° C. | | | |

| | | | | | | |
|---|---|---|---|---|---|---|
| 6 | 5 g & | 1. | 4-(TBS- | n-BuLi dropwise | Aqueous | Yield = 71% |
| | 7.5 g | | oxymethyl)bromobenzene | over 25 min (A), | work-up | (after following |

| | | | |
|---|---|---|---|
| | (1.0 eq), tBu₂SiF₂ (1.15 eq.), | 35 min (B) | step and |
| | THF, 0-5° C. Add RM. | (2.5M/hexanes; | purification) |
| 2. | 2 h at 0-5° C. (A), 18 h | 1.15 eq.) | |
| | warming to rt (B) | | |

1. A method of preparing a compound of formula (1) comprising reacting a compound of formula (2) with an alkyllithium reagent of formula (3) and a compound of formula (4): 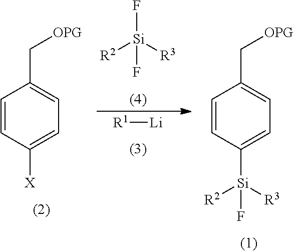
wherein PG is a protecting group;
X is Br, Cl or l;
R² and R³ are independently linear or branched C₁₋₁₀ alkyl;
R¹ is linear C₁₋₆ alkyl; and
wherein the alkyllithium reagent of formula (3) is added to a mixture comprising the compounds of formula (2) and (4).

2. The method of claim 1, wherein the alkyllithium reagent is n-butyllithium.

3. The method of claim 1 or claim 2, wherein PG is an acid-labile protecting group.

4. The method of claim 3, wherein PG is tert-butyldimethylsilyl(TBDMS).

5. The method of any one of claims 1 to 4, wherein X is Br.

6. The method of any one claims 1 to 5, wherein R² and R³ are both tert-butyl.

7. The method of any one of claims 1 to 6, wherein the mixture comprising the compounds of formula (2) and (4) is maintained at a temperature of between −5° C. and 10° C. during addition of the alkyllithium reagent.

8. The method of any one of claims 1 to 7, additionally comprising a step of deprotecting the compound of formula (1) using an acid to obtain a compound of formula (5):

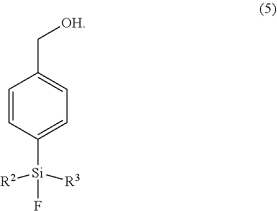

9. The method of claim 8, wherein the acid is hydrochloric acid.

10. The method of claim 8 or claim 9, additionally comprising a step of oxidising the compound of formula (5) to obtain a compound of formula (6):

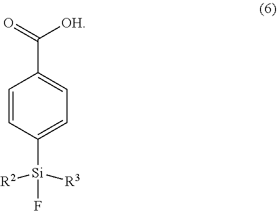

11. The method of claim 10, wherein the oxidizing agent is potassium permanganate.

12. The method of claim 10 or claim 11, further comprising a trituration purification step.

13. The method of claim 12, wherein the compound of formula (6) is used in the manufacture of a radiopharmaceutical.

14. The method of claim 13, wherein the radiopharmaceutical is a ligand-SIFA-chelator conjugate, comprising:
(a) one or more ligands which are capable of binding to prostate-specific membrane antigen (PSMA);
(b) a silicon-fluoride acceptor (SIFA) moiety which comprises a covalent bond between a silicon and a fluorine atom; and
(c) one or more chelating groups, optionally containing a chelated nonradioactive or radioactive cation.

15. The method of claim 14, further comprising an 18F fluorine exchange step.

16. The method of claim 14 or 15, wherein the chelator comprises a chelated nonradioactive Ga³⁺ cation.

17. The method of any one of claims 14 to 16, wherein the ligand-SIFA-chelator conjugate is selected from:

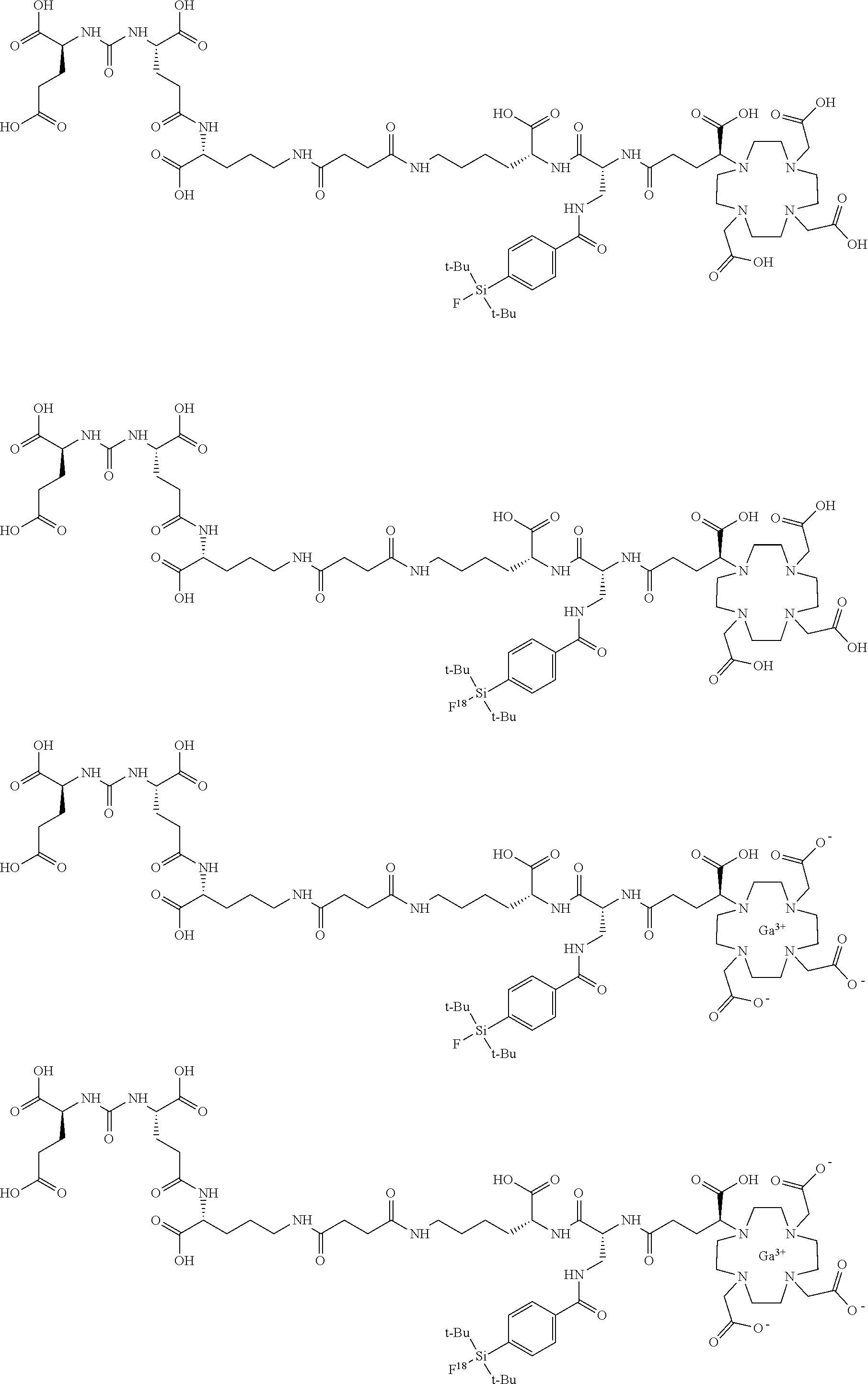

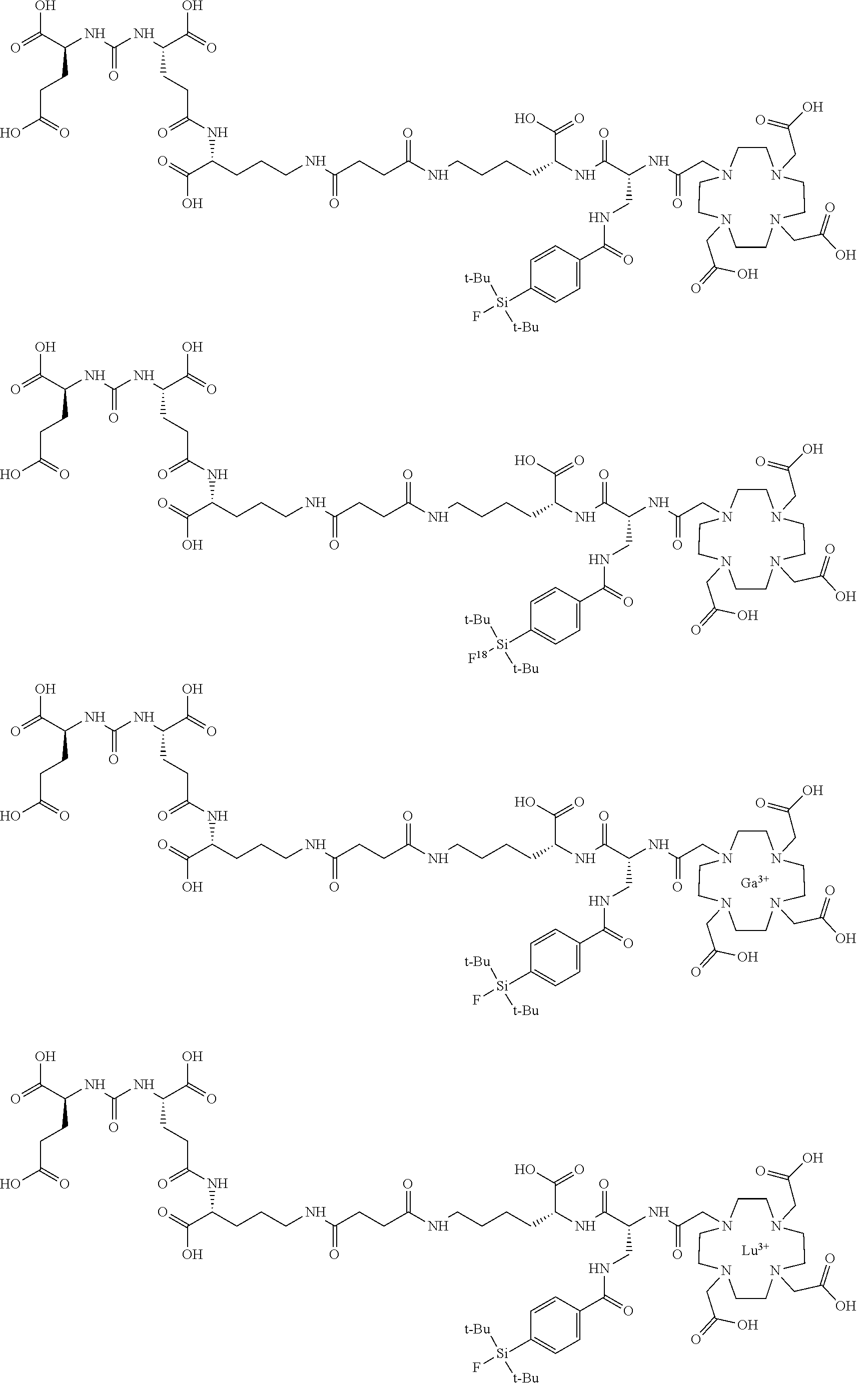

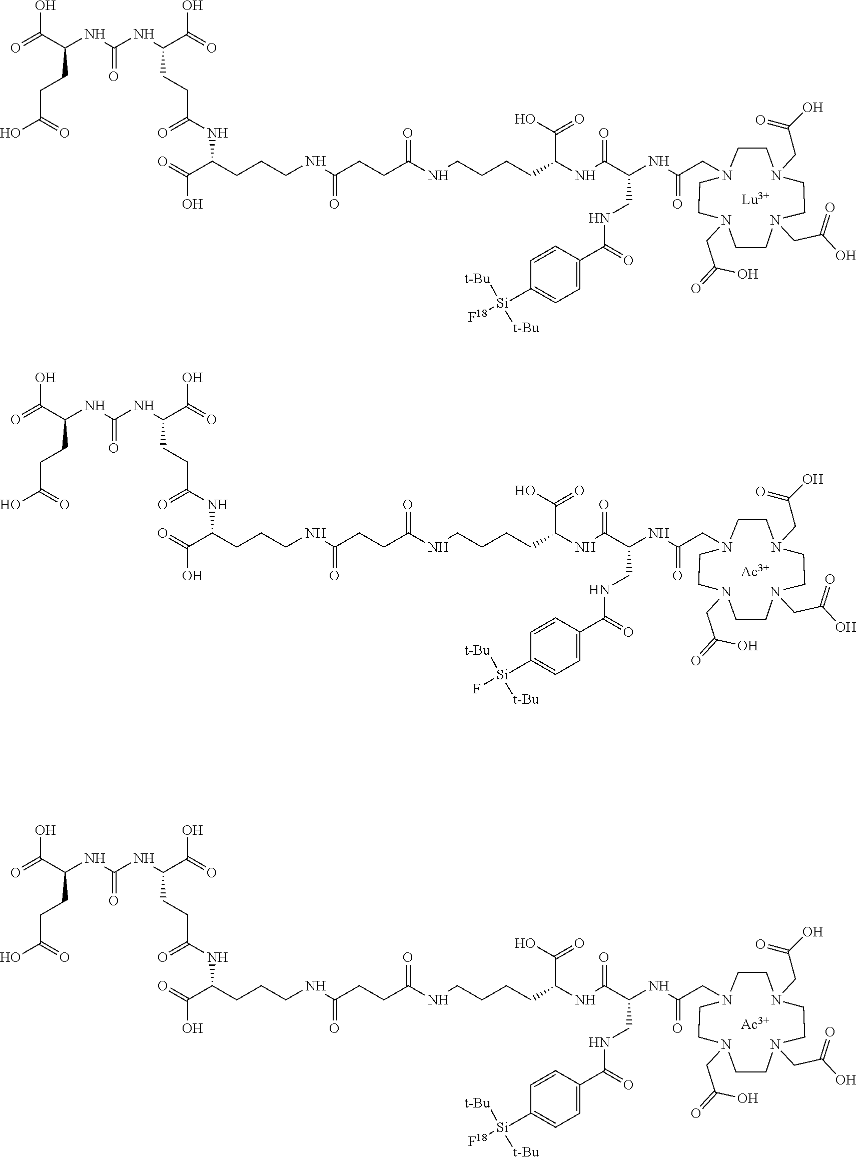

18. A composition comprising a ligand-SIFA-chelator conjugate selected from: 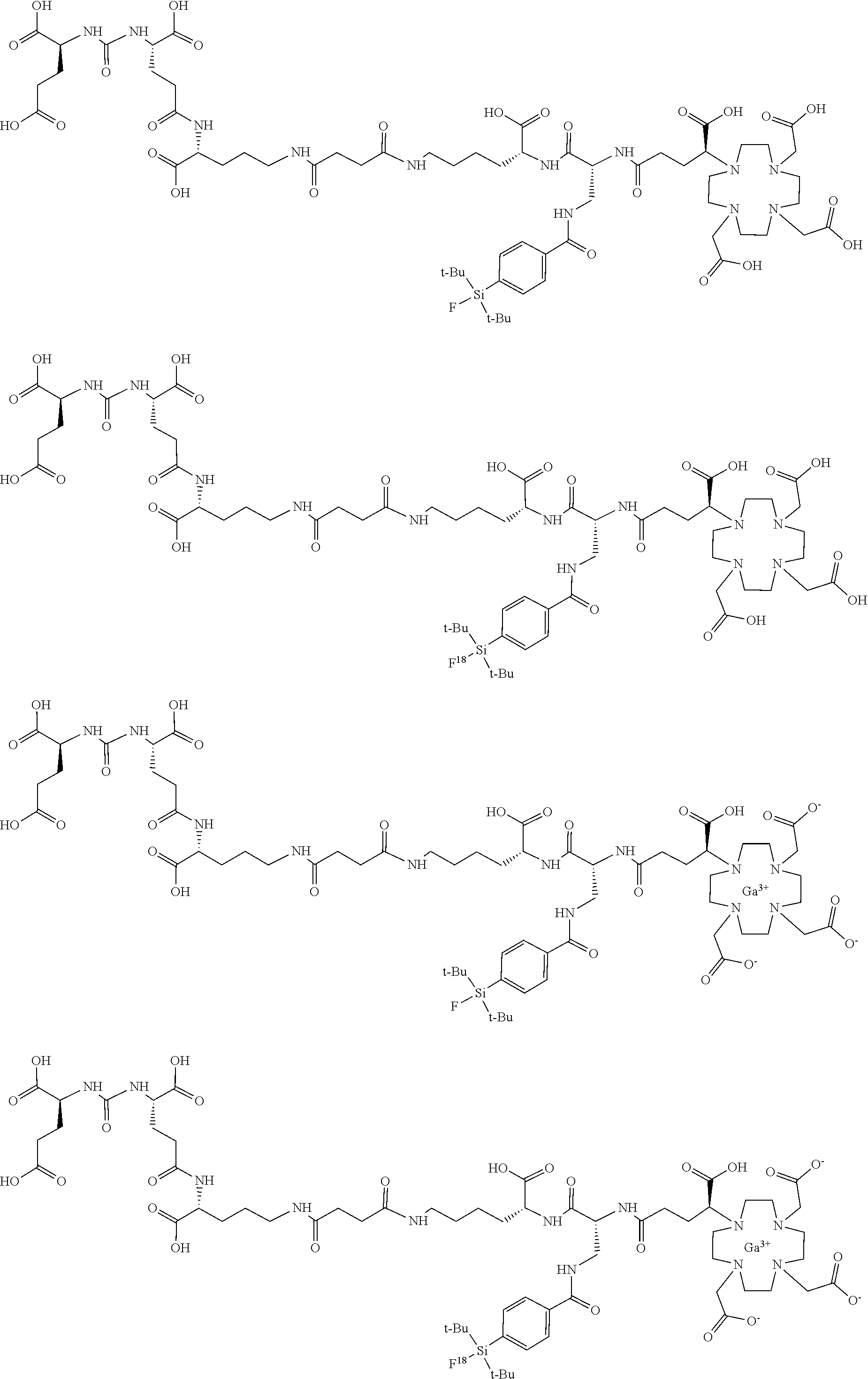 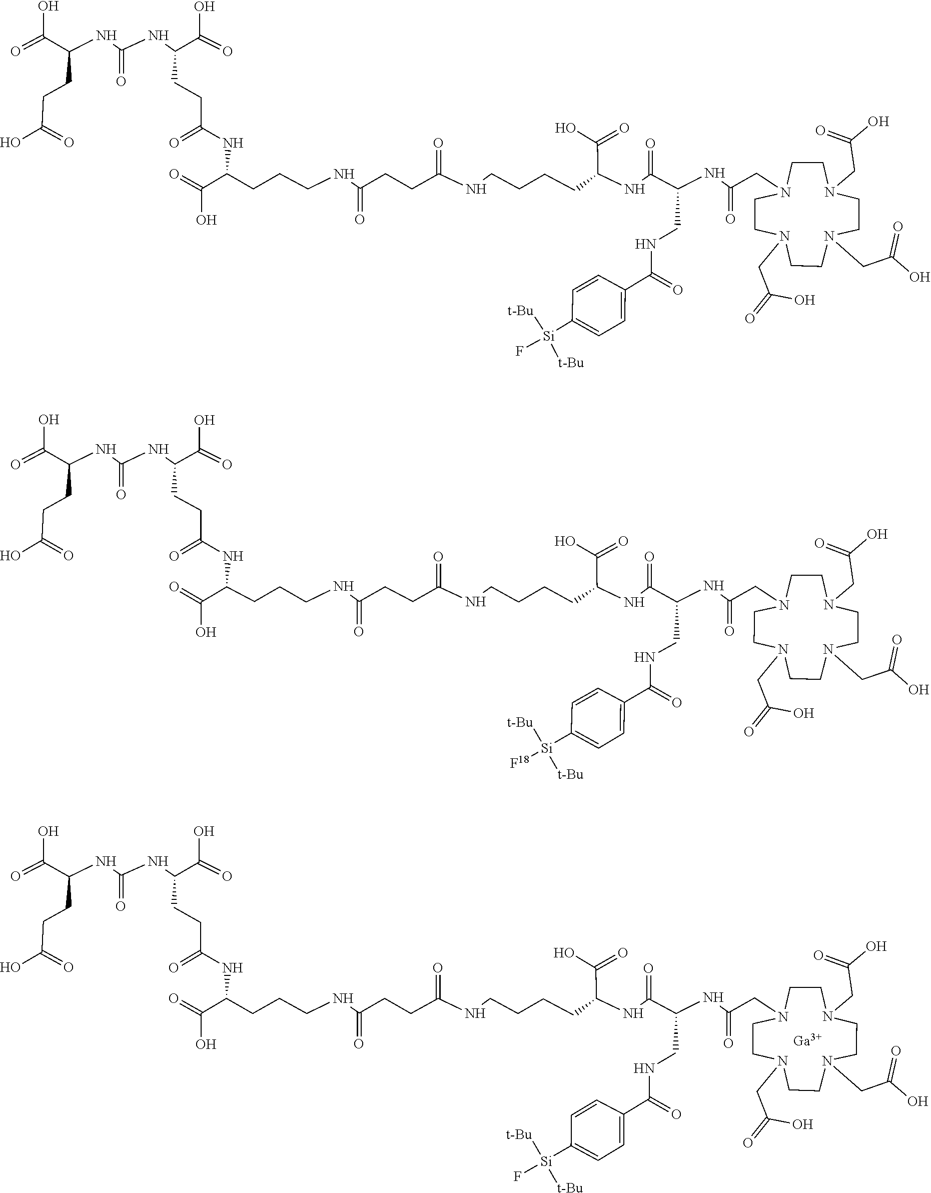 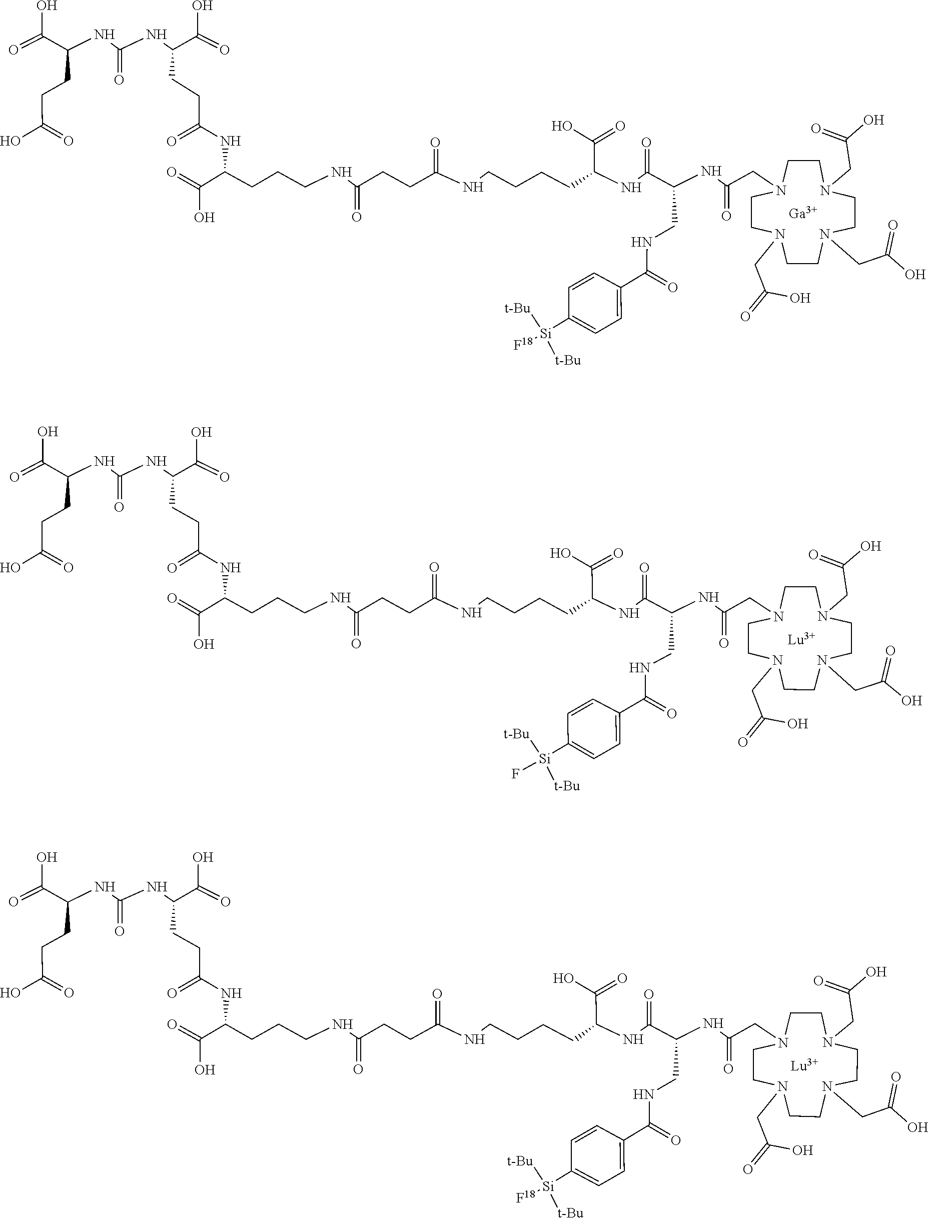 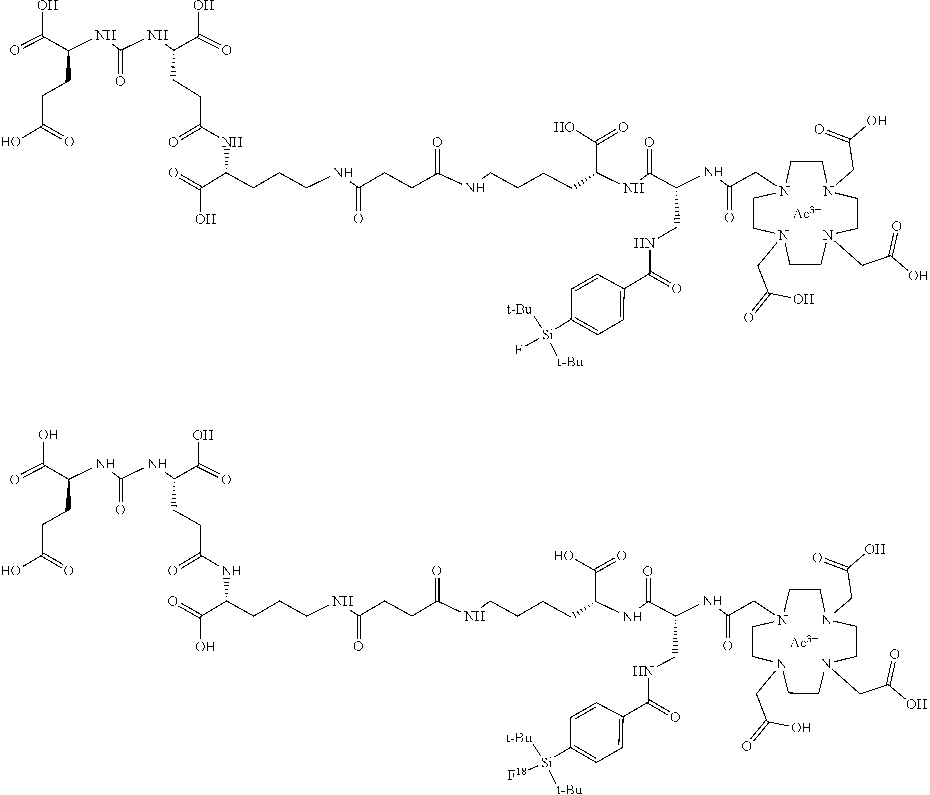
wherein said compound is prepared using the method of claim 12, and wherein said composition comprises no more than 0.1% (w/w) of a compound comprising a group of the formula:

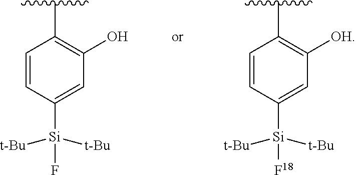

19. A composition according to claim 18, for use as a cancer diagnostic or imaging agent or in the treatment of cancer.

20. A composition according to claim 18, for the diagnosis, imaging or prevention of neoangiogenesis/angiogenesis.

21. A composition according to claim 18, for use as a cancer diagnostic or imaging agent or for use in the treatment of cancer wherein the cancer is prostate, breast, lung, colorectal or renal cell carcinoma.
